(19) United States
(12) Patent Application Publication
(10) Pub. No.: US 2010/0290997 A1
(43) Pub. Date: Nov. 18, 2010

(54) Imaging Agents and Methods

(76) Inventors: Chun Li, Missouri City, TX (US); Wei Wang, Sugar Land, TX (US); Marites P. Melancon, Houston, TX (US); Juri G. Gelovani, Houston, TX (US); Jeffrey Myers, Houston, TX (US)

Correspondence Address: FULBRIGHT & JAWORSKI L.L.P., 600 CONGRESS AVE., SUITE 2400, AUSTIN, TX, 78701, US

(21) Appl. No.: 12/227,185

(22) PCT Filed: May 11, 2007
(86) PCT No.: PCT/US07/68783

§ 371 Date: Jun. 22, 2010

Related U.S. Application Data
(60) Provisional application No. 60/747,180, filed on May 12, 2006.
(60) Provisional application No. 60/819,297, filed on Jul. 7, 2006.

(51) Int. Cl. A61K 49/06; C07D 403/10; C12Q 1/02; A61K 49/00

(52) U.S. Cl. 424/9.3; 548/455; 435/29; 424/9.1

(57) ABSTRACT

The disclosure includes a composition including a poly(L-glutamic acid) and a NIRF dye. It also includes a method including providing to a plurality of cells an imaging agent including poly(L-glutamic acid), a NIRF dye and then imaging the cells to detect the imaging agent. It further includes a dual functional contrast agent including an MRI agent conjugated with an optical imaging agent. A method of detecting cancer is provided including injecting a dual functional contrast agent into a patient and performing both an MRI and an optical scan. The presence of the agent may indicate cancer. A method of detecting cancer by injecting PG-DTPA-Gd-NIR813 into a patient, then detecting the presence or absence of Gd in a cell or tissue of the patient and detecting the presence or absence of NIR813 in a cell or tissue of the patient is provided. The presence of Gd and NIR813 may indicate cancer.

### RELATED APPLICATIONS

This application claims priority to U.S. Provisional Patent Application Ser. No. 60/747,180, filed May 12, 2006, and entitled “Imaging Agents and Methods,” the contents of which are incorporated herein in their entirety by reference.

This application also claims priority to U.S. Provisional Patent Application Ser. No. 60/819,297, filed Jul. 7, 2006, and entitled “Dual Modality Mr/Optical Imaging With A Macromolecular Contrast Agent,” the contents of which are incorporated herein in their entirety by reference.

### STATEMENT OF GOVERNMENT INTEREST

This disclosure was developed at least in part using funding from the National Institute of Health, Grant Numbers R01 EB000174, R01 EB003132, and U54 CA90810, and the National Cancer Institute, Grant Number R01 CA119387. The U.S. government may have certain rights in the invention.

### TECHNICAL FIELD

The present disclosure relates to medical imaging and imaging agents. Embodiments relate to near-infrared fluorescence imaging and imaging agents. Other embodiments relate to dual modality imaging, such as magnetic resonance and optical, e.g. near-infrared fluorescence, imaging.

### BACKGROUND

Near-infrared fluorescence (NIRF) optical imaging is currently under development in several laboratories as a diagnostic modality that potentially allows imaging of biologic systems at the cellular and molecular level. In the NIRF wavelength region (700-900 nm), light can travel several centimeters owing to the tissue's ability to multiply scatter light and to the relatively low absorbance associated with water, fat, hemoglobin and other less contributing biological molecules. In addition, endogenous fluorescence is minimal in the NIRF range.

Successful translation of NIRF optical imaging into clinical use requires advances in several fronts, including development and validation of fluorescence-based contrast agents. One approach towards practical use of optical imaging agents is the development of “smart” probes, or molecular beacons that change their optical properties on interaction with specific molecular processes.

Cathepsin B (CB) are known to be important in normal tissue remodeling, but also known to play critical roles in many diseases, such as arthritis, atherosclerosis and cancer. Elevated level of CB have been found in tumors and shown to correlate well with their invasive and metastatic profiles in both experimental cancer models and in human malignancies.

Poly(L-glutamic acid) (PG) has been used as a macromolecular carrier for drug delivery, specifically to target cancer. PG-drug conjugates have been shown to be more potent and less toxic than their parent unconjugated drugs. In vivo degradation of PG by cathepsin B (CB) has been linked to the increased site-specific delivery of anticancer drugs and enhanced antitumor activity of such PG-drug conjugates as PG-paclitaxel (Xyotax®) and PG-camptothecin (CT2003). In clinical studies with PG-paclitaxel conjugated, Xyotax®, significantly increased antitumor activity was noted in women with lung cancer with in elevated estrogen receptors, which in turn has been related to increased CB activity. Although the degradation of PG by CB has been extensively studied in vitro using either purified CB or cell lysates, studies of the kinetics of in vivo degradation of PG in various tissues in live animal have not be possible because of the lack of suitable technology.

Determining in vivo degradation of biomaterials and polymeric drug is traditionally carried out by analyzing the appearance of degradation products in the target tissues. This method requires killing animals at each time point so that tissues can be removed from the animals. The degradation products are identified often using tedious purification scheme in combination with several detection methods including UV/Vis spectroscopy and mass spectroscopy. For example, a recent report confirmed monoglutamyl-2′-TXL and diglutamyl-2′TXL as the major intracellular metabolites of Xyotax using LC-MS technique, and the degradation of the polymer is correlated to its enhanced antitumor activity. Imaging technology for monitoring degradation of PG-based anticancer drugs in living animals is highly desirable because such method may potentially facilitate devising strategies for individualized therapy with Xyotax and non-invasive monitoring of treatment response to Xyotax treatment.

Sentinel lymph node (SLN) mapping is a method of determining whether cancer has metastasized beyond the primary tumor and into the lymph system. Traditionally, lymph node (LN) status has been assessed using clinical palpation and radiographic imaging of macroscopically enlarged nodes. Unfortunately, this approach is not highly accurate and frequently misses early LN metastases. Recently, a new technique to identify early lymph node metastases—lymphatic mapping with sentinel node biopsy (LSNB)—has been adopted to evaluate microscopic regional LN metastases in patients with melanoma, gastrointestinal, or breast cancer who have no clinical nodal involvement. In this technique, radiolabeled particles, sulfur colloid particles, and blue dye are injected and their localization to the SLN was visualized by naked eyes and with the help of hand-held gamma counter. While LSNB has reduced morbidity of regional staging by avoiding unnecessary removal of the entire nodal basins, LSNB still requires multiple injections, an invasive surgical procedure, and up to two weeks of waiting to determine whether or not cancer cells have spread. The radionuclide technique is also limited by exposure to ionizing radiation and the low spatial and temporal resolution.

To overcome these limitations, several contrast agents for MRI have been designed to provide a minimally invasive, fast, and sensitive method to detect SLN. MRI is being used to characterize lymph nodes abnormalities in cancer patients because of its excellent spatial and temporal resolution. Published techniques have used intravenous and interstitial injection of contrast agents to determine the metastatic status of lymph node. This includes using dextran-stabilized iron oxide crystals have helped to distinguish between normal and tumor-bearing nodes or reactive and metastatic nodes with magnetic resonance imaging; using iron oxide nanoparticles for strong negative enhancement to identify lymph nodes; and Gd-DTPA dendrimer-based contrast agent which gives T1-positive contrast enhancement of the lymphatic ducts and lymph nodes in mice.

For example, Gd-DTPA labeled polyglucose significantly enhanced T1-weighted signal intensity of normal but not metastatic nodes in a rabbit model in regional nodes 24 hr postinjection. Harika L, Weissleder R, Poss K, et al. MR lymphography with a lymphotropic T1-type MR contrast agent: Gd-DTPA-PGM. Magn Reson Med 1995; 33:88-92, MR lymphography performed using dendrimers visualized regional draining lymph nodes better than small molecular weight contrast agents. Kobayashi H, Kawamoto S, Sakai Y, et al. Lymphatic drainage imaging of breast cancer in mice by micro-magnetic resonance lymphangiography using a nano-size paramagnetic contrast agent. J Natl Cancer Inst 2004; 96:703-708. However, the technique is difficult for real time visualization, which limits the use of MRI alone in SLN mapping. Soltesz E G, Kim S, Laurence R G, et al. Intraoperative sentinel lymph node mapping of the lung using near-infrared fluorescent quantum dots. Ann Thorac Surg 2005; 79:269-277; discussion 269-277, all incorporated by reference herein.

Optical imaging is a relatively new modality that provides distinctly new diagnostic capabilities while complementing conventional imaging modalities. Some advantages of optical imaging methods include the use of non-ionizing radiation, high sensitivity with the possibility of detecting micron-sized lesions, capability of continuous data acquisition for real time monitoring during surgery, and the development of potentially cost-effective equipment. It also provides flexibility in the mode of chromophore excitation (broadband light source, modulated light, continuous wave or pulsed laser and signal detection (transillumination or reflectance, and scattering, absorption or fluorescence modes). Optical imaging methods can be completely non-invasive, especially when endogenous chromophores are used; minimally invasive, when contrast agents are injected; or invasive, when used in conjunction with surgical procedures or catheterization. For example, quantum dots (QD) have been used to map sentinel lymph nodes in mice and pigs.

Quantum dots (QD) have been used as NIRF agents to identify SLN. Questions remained to be addressed before QD-based optical imaging techniques are translated into human studies. First, optical imaging is difficult for visually identifying deeper SLN owing to light attenuation. Second, potential toxicities of QD, which is made of toxic heavy metal ions such as cadmium, telluride, selenide, cause considerable concern. Although coating with a layer of biocompatible materials on the surface of QD reduces the side effects of QD, long-term effects of QD in the body remains to be studied.

Recently, it has been recognized that combination of MRI and optical imaging can lead to the development of new approaches which will bridge the gaps in resolution and depth of imaging between these two modalities and at the same time provide complimentary anatomic, functional and molecular information. The combination of MRI with near-infrared (NIR) optical imaging was evaluated in tumor models and in the detection of cancer in human pilot clinical studies. In those experiments, MRI was used to obtain precise anatomic information on the location of tissue structures that were probed optically.

### SUMMARY

According to one embodiment, the invention relates to a composition having the formula:

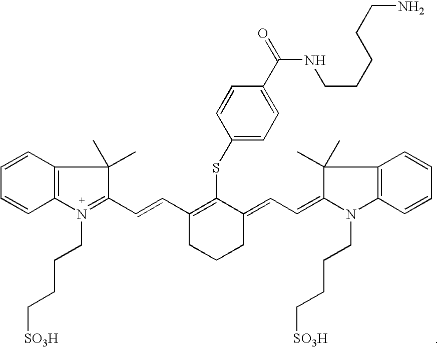

According to another embodiment, the invention relates to a composition including a poly(L-glutamic acid) and a NIRF dye.

According to another embodiment, the invention relates to a method including providing to a plurality of cells an imaging agent including poly(L-glutamic acid), a NIRF dye and then imaging the cells to detect the imaging agent.

According to another embodiment, the invention includes a dual functional contrast agent. This agent may include an MRI agent comprising Gadolinium conjugated with an optical imaging agent.

According to another embodiment, the invention includes a method of detecting cancer. This method include injecting a dual functional contrast agent into a patient The dual functional contrast agent may include an MRI agent conjugated with an optical imaging agent. The method may also include performing an MRI scan in the patient to detect the presence or absence of the contrast agent and performing an optical scan on the patient to detect the presence or absence of the contrast agent. The presence of the contrast agent in a cell or tissue may correlates with the presence of cancer in the cell or tissue.

Finally, according to another embodiment, the invention may include a method of detecting cancer by injecting PG-DTPA-Gd-NIR813 into a patient, then detecting the presence or absence of Gd in a cell or tissue of the patient and detecting the presence or absence of NIR813 in a cell or tissue of the patient. The presence of Gd and NIR813 in a cell or tissue of the patient may be indicative of cancer.

### BRIEF DESCRIPTION OF THE DRAWINGS

Some specific example embodiments of the disclosure may be understood by referring, in part, to the following description and the accompanying drawings. The following figures form part of the present specification and are included to further demonstrate certain aspects of the present description. The patent or application file contains at least one drawing executed in color. Copies of this patent or patent application publication with color drawing(s) will be provided by the Office upon request and payment of the necessary fee.

FIG. 1 shows the structures of NIR813 and PG-NIR813 conjugates.

FIG. 2 shows the fluorescence spectra of IR783 and NIR813. The excitation/emission wavelengths are 766/798 nm for IR783 and 766/813 nm for NIR813. Measurements were made in a methanol solution. NIR813 has a longer emission wavenumber and greater Stokes shift (47 nm) than IR783 (32 nm).

FIG. 3 shows images illustrating the effect of NIR813 loading on the quenching efficiency of PG-NIR813. A. NIRF imaging acquired after 1 h of incubation at room temperature. B. Fluorescence intensity as a function of NIR813 loading. Each well contained 100 μL PG-NIR813 at a final concentration of 10 μM equivalent NIR813 molecules. The images were acquired and analyzed using a Li-Cor Odyssey imaging system. NIR813 loading on PG (17 KDa) is expressed as a percentage of the number of repeating units in PG.

FIG. 4 shows images illustrating the effect of NIR813 loading on degradation of PG-NIR813 and re-activation of fluorescence signal by cathepsin B. A. NIRF imaging. B. Fluorescence intensity as a function of incubation time. Each well contained 0.4 unit/mL cathepsin B in 100 μL sodium acetate buffer (20 μM, pH 5). Wells were incubated without PG-NIR813 (C1) or with PG-NIR813 (10 μM eq. NIR813) containing 15% (C2), 10% (C3), 8.3% (C4), 4.4% (C5), and 1% (C6) of NIR813 dye. The wellin column 7 contained cathepsin B and NIR813 (10 μM) as a control.

FIG. 5 is a comparison of in vitrodegradation of L-PG-NIR813 (abbreviated as PG-NIR813) and D-PG-NIR813. A. NIRF imaging. B. Fluorescence intensity as a function of incubation time. Each well contained 0.8 unit/mL cathepsin B in 100 μL sodium acetate buffer (20 μM, pH 5). Wells were incubated with 10 μM eq. NIR813 of D-PG-NIR813 (C1) or PG-NIR813 (C2). Both conjugates contained 10% of NIR813 dye.

FIG. 6 shows images illustrating the effect of cathepsin B concentration on the activation of PG-NIR813 (8.3% dye loading, 20 μM eq. NIR813). PG-NIR813 (17 KDa) was incubated with cathepsin B at room temperature for various times for up to 24 hr. Fluorescence intensity increased with increasing concentration of cathepsin B and increasing incubation times.

FIG. 7 is a graph showing the degradation kinetics of PG-NIR813 (8.3% loading, 17 KDa) by cathepsin B. Product concentrations were derived from the standard curve produced with the unconjugated NIR813. Non-linear fits of all data sets gives the initial velocities, which were used to generate Michaelis-Menten graph.

FIG. 8 are Michaelis-Menten graphs for PG-NIR813 (17 KDa) and PG-NIR813 (56 KDa). Higher molecular weight conjugate degraded at a slower rate.

FIG. 9 shows images illustrating inhibition of PG-NIR813 degradation by selective cathepsin B inhibitor (inhibitorII). (Top): NIRF images taken 21 h after incubation of PG-NIR813 conjugate (8.3% loading, 10 μM eq. NIR813) in the presence (bottom panel) and absence (top panel) of cathepsin B (0.2 unit/mL). Microwellsin the bottom panel were added increasing concentrations of cathepsin B inhibitor II. (Bottom): Fluorescence signal intensity as a function of inhibitor concentration.

FIG. 10 shows images illustrating the specificity of PG-NIR813 degradation by proteinases. PG-NIR813 (10% loading, 40 μMeq. NIR813) was incubated with cathepsin B (0.04 unit), cathepsin D (0.08 unit), cathepsin E (0.08 unit), or MMP-2 (50 ng) at 37° C. over a period of 24 h. The buffer and pH value of the buffer used in the degradation studies were selected according to manufacturer provided procedures. Fluorescence intensity only increased with the use of cathepsin B. Data are presented as an average of duplicate experiments.

FIG. 11 shows images illustrating the degradation of PG-NIR813 (10% loading) by U87 cells in vitro. Cells were seeded (1×10 6 cells) in 96-well plate for 24 h. The cells were then treated with PG-INIR813 under the following conditions: (A). 0.1 μMPG-INIR813 for 24 h without changing culture media; (B) fresh culture media followed by 0.1 μMPG-NIR813 for 24 h; (C) 24 incubation without PG-NIR813. Images were taken with culture media.

FIG. 12 shows images illustrating the in vivo degradation of PG-NIR813 (10% loading, MW 17K). NIRF images were acquired at various times after intravenous injection of PG-NIR813 at a dose of 10 nmol eq. NIR813 per mouse. One mouse was killed at 4 h after NIRF dye injection to verify tissue distribution. PG-NIR813 was primarily degraded in the liver was cleared from the body through GI tract.

FIG. 13 shows images illustrating the in vivo degradation of PG-NIR813 (10% loading, MW 17K) in human U87/TGL glioma inoculated in the brain. NIRF images were acquired at 24 hr after intravenous injection of PG-NIR813 at a dose of 50 nmol eq. NIR813 per mouse. The presence of tumors in the brain was confirmed by chemoluminescent optical imaging of luciferase activity in U87/TGL tumors. Fluorescence signal was detected only the brain of mice injected with L-PGNIR-813 but not in mice injected with non-degradable D-PG-NIR813.

FIG. 14 is an image showing fluorescence spectrum of PG-DTPA-Gd-NIR813 (1% loading) and NIR813. The polymeric conjugate with low NIR813 dye loading (<1%) retained most of the fluorescence signal with minimal quenching effect.

FIG. 15 are images showing PG-DTPA-Gd-NIR813 drained to the sentinel lymph nodes soon as 5 min after subcutaneous injection at the front paw (arrow). The fluorescence signal co-localized with isosulfan blue dye visualized under bright light (arrow heads). Isosulfan blue is used as a gold standard for SLN mapping.

FIG. 16 are representative microphotography images of H&E stained section and fluorescence micrography of the same section from a dissected lymph node. Fluorescence signal was detected only in the lymph node (pseudocolor, red) but not in the adjacent muscle tissue (red).

FIG. 17 shows comparison of NIRF optical images acquired 1 hr after subcutaneous injection of PG-DTPA-Gd-NIR813 at doses of 0.02 mmol Gd/kg (48 nmol eq. NIR813) (A) and 0.002 mmolGd/kg (4.8 nmoleq. NIR813) (B). SLN (arrow heads) were detected at both doses.

FIG. 18 shows comparison of MR images acquired at different times after subcutaneous injection of PG-DTPA-Gd-NIR813 at doses of 0.02 mmol Gd/kg (A) and 0.002 mmolGd/kg (4.8 nmoleq. NIR813) (B). SLN (arrow heads) were clearly delineated at both doses.

FIG. 19 shows the reaction scheme for the synthesis of IR783-NH2 and PG-Benz-DTPA-Gd-IR783.

FIG. 20 shows a fluorescence emission spectra of PG-benz-DTPA-Gd-IR783 (in water) and IR783-NH2 (in ethanol/water). Plot of intensity (arbitrary units, AU) vs wavelength (nm) depicting PG-benz-DTPA-Gd-IR783 and IR783-NH2 fluorescence after excitation at 765 nm.

FIG. 21 shows images of co-localization of PG-benz-DTPA-Gd-IR783 with isosulfan blue dye. Male, athymic nude mice were injected subcutaneously with 4.8 nmol IR783/mouse using PG-benzDTPA-Gd-IR783 in the left paw, the pre-injection of PG-benzDTPA-Gd-18783 overlay image of white light and NIR fluorescence, and the 5 min post-injection overlay of white light and NIR fluorescence. The arrows indicate the putative axiliary and branchial lymph nodes. Fluorescence images have identical exposure times and normalization, image of the mouse after the injection of 1% isosulfan blue at the same location as the contrast agent, and after 5 minutes with the exposure of the actual lymph nodes. Isosulfan blue and PG-benzDTPA-Gd-IR783 were localized in the same lymph nodes: resected lymph nodes for histology

FIG. 22 shows images of lymph node (top row) and muscle (bottom row) after resection. Hematoxylin and eosin (H&E) staining (left) confirmed the identity of the lymph node, while the near infra-red fluorescence confirmed the contrast agent uptake of PG-benzDTPA-Gd-IR783 into the LN. Overlapping the DIC and fluorescence indicates the localization of PG-benzDTPA-Gd-IR783 within the LN. Muscle does not have fluorescence.

FIG. 23 shows in vivo optical images of the axial and branchial lymph nodes in athymic nude mice before and after the injection of PG-benz-DTPA-Gd-IR783 at 0.02 mmol Gd/kg and 0.002 mmol Gd/kg. NIR fluorescence images have identical exposure times and normalizations. Also, these lymph nodes were excised for histological evaluations.

FIG. 24 shows T1-weighted axial MR images of PG-benz-DTPA-Gd-IR783 at (A) 0.02 mmol Gd/kg and (B) 0.002 mmol Gd/kg. MR signal intensity increases with increasing time.

FIG. 25 is a graph of the time course of lymph node enhancement using 0.02 mmol Gd/kg and 0.002 mmol Gd/kg of PG-benzDTPA-Gd-IR783. This graph indicates higher SI in higher concentration than low.

FIG. 26 illustrates a reaction scheme for the synthesis of NIR813 (FIG. 26A) and PG-DTPA-Gd-NIR813 (FIG. 26B).

FIG. 27 shows the fluorescence emission spectra of NIR813 (1 μM, in methanol) and PG-DTPA-Gd-NIR813 contrast agent (1 μM, in water). The solutions were excited at 766 nm.

FIG. 28A-D show NIRF images in mice demonstrating co-localization of PG-DTPA-Gd-NIR813 and isosulfan blue dye in sentinel lymph nodes. Each mouse was injected subcutaneously with PG-DTPA-Gd-NIR813 contrast agent (10 μL, 4.8 nmol eq. NIR813/mouse) in the left paw. FIG. 28A shows a pre-contrast overlay of white light and NIRF images. FIG. 28B shows an overlay of white light and NIRF images 5 min post-contrast agent injection. The arrows indicate the putative sentinel lymph nodes. FIG. 28C shows photography of the same mouse showing the same lymphatic nodes (arrows) stained blue by isosulfan blue. FIG. 28D shows fluorescence signal in and around resected lymph nodes. FIGS. 28E-H show microphotographs of representative resected lymph nodes to evaluate the uptake of PG-DTPA-Gd-NIR813 in the lymph nodes. FIG. 28E shows an H&E stained tissue section. FIG. 28F shows a DIC image. FIG. 28G shows an NIRF image. FIG. 28H shows an overlay of the DIC and NIRF images. The NIRF signal is pseudocolored green, and the DIC pseudocolored red. Original magnification: 50×.

FIG. 29 shows dual MR/optical imaging of the axial and branchial lymph nodes in athymic nude mice. FIGS. 29A-D are NIRF images. FIG. 29A is a pre-contrast overlay of white light and NIRF images. FIG. 29B is an overlay of white light and NIRF images 1 hr after injection of PG-DTPA-Gd-NIR813 (0.002 mmol Gd/kg). FIG. 29C is an NIRF image of the same mouse without skin. FIG. 29D shows fluorescence signal of resected lymph nodes. FIGS. 29E-F show representative T1-weighted axial MR images at different times. In FIG. 29E PG-DTPA-Gd-NIR813 was injected at a dose of 0.02 mmol Gd/kg and in FIG. 29F at a dose of 0.002 mmol Gd/kg. The arrows indicate sentinel nodes.

FIG. 30 shows the time course of lymph node enhancement at doses of 0.02 mmol Gd/kg and 0.002 mmol Gd/kg of PG-DTPA-Gd-NIR813. All data were expressed as mean±SD.

FIG. 31 shows visualization of cervical lymph nodes after interstitial injection of PG-DTPA-Gd-NIR813 (0.02 mmol Gd/kg) into the tongue of a normal mouse (FIGS. 31A-E) and a mouse with a human DM14 squamous carcinoma tumor grown in the tongue (FIGS. 6F-J). FIGS. 31A&F show T1-weighted coronal images acquired 2 hr after contrast injection. FIGS. 31B&G show an overlay of white light and NIRF images 24 hr after contrast injection. FIGS. 31C&H show NIRF images of mice without skin. FIGS. 31D&I show NIRF images of resected lymph nodes. The arrows indicate sentinel nodes and arrowhead indicates the primary tumor. FIGS. 31E&J show microphotographs of H&E stained lymph node sections. FIG. 31K shows microphotographs of H&E stained tongue section indicating the presence of micrometastases, presumably in-transit metastases in the lymphatic duct.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DETAILED DESCRIPTION

The present disclosure provides, according to certain embodiments, a NIRF dye having the following structure:

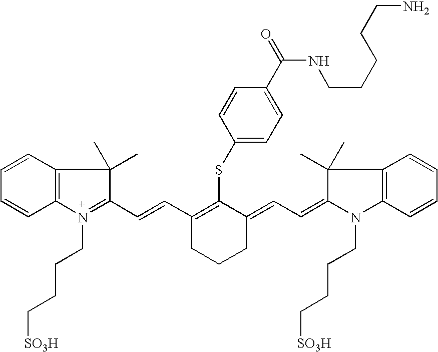

This dye is referred to as NIR813. This dye has longer excitation and fluorescence wavelengths and a greater Stokes shift (difference between the excitation wavelength and emission wavelength) than Cy5.5. This means images acquired using imaging agents that comprise NIR813 can penetrate deeper into the tissues and can have less interference from the excitation light with appropriate filter sets as compared to those acquired with Cy5.5 derivatives.

The present disclosure provides, according to certain embodiments, a composition comprising NIR813. According to certain embodiments such compositions may be referred to as imaging agents and may comprise poly(L-glutamic acid) and a NIRF dye, such as for example, NIR813 and IR783.

In general, such imaging agents are present in a quenched (i.e., inactive) state in aqueous solution but becomes dequenched (i.e., activated) when cleaved, for example, upon exposure to a proteinases like CB. Accordingly, these imaging agents may be used, among other things, for in vivo molecular optical imaging.

In other embodiments, the imaging agent may further comprise a paramagnetic metal chelate (e.g., Gd-DTPA). The DTPA-Gd is conjugated to PG so that the conjugate can be used as an MRI contrast agent in addition to its NIRF properties. Accordingly, these imaging agents may be used to detect SLN using both optical and MR imaging.

One example of an imaging agent comprises poly(L-glutamic acid) and NIR813 as the NIRF dye. This imaging agent may be referred to as PG-NIR813 and has the following structure:

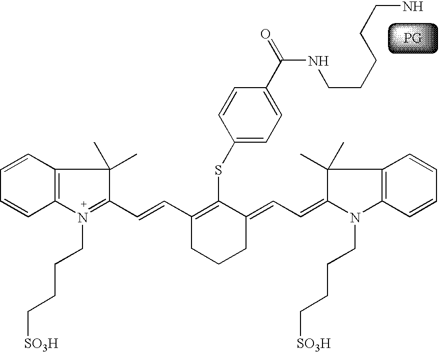

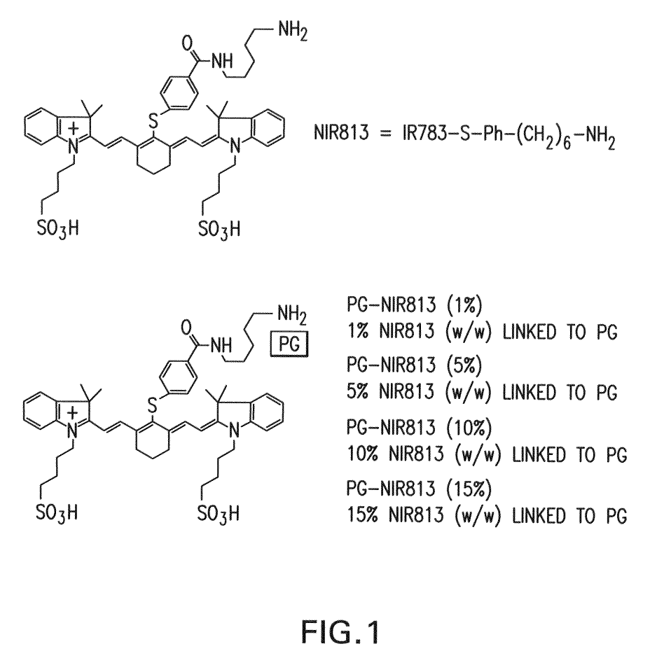

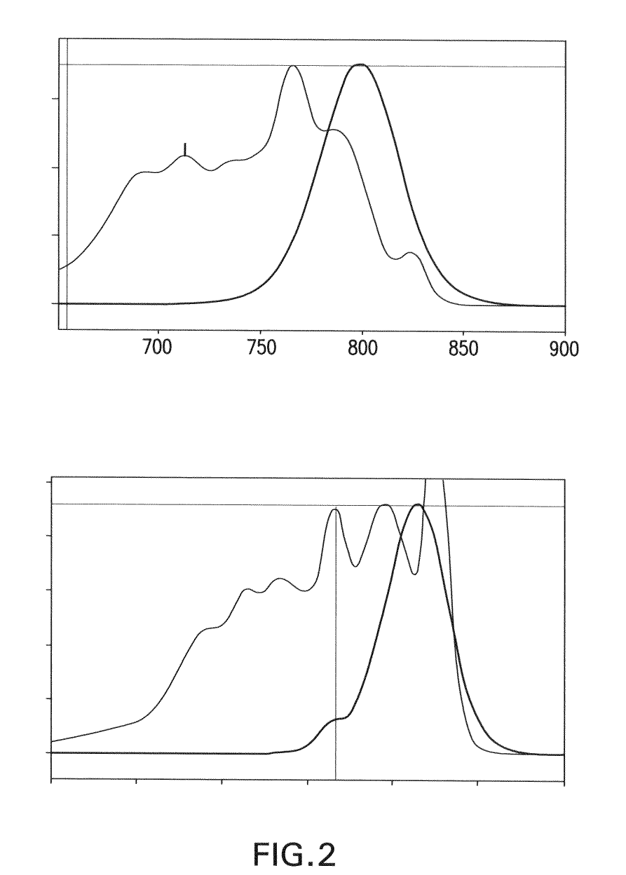

The NIR813 may be present at from about 1% w/w linked to PG to about 15% w/w linked to PG. (See FIG. 1). PG-NIR813 has excitation and emission wavenumbers of 766 nm and 813 nm, respectively. The long wavenumber allows deeper penetration into the tissues and has less interferences from autofluorescence (i.e., signal coming from endogenous fluorophores). Such imaging agents may be used, among other things, for in vivo molecular optical imaging of proteinases like CB at diseased sites, and in vitro assays of CB activity in biological samples.

One example of an imaging agent comprises poly(L-glutamic acid), NIR813 as the NIRF dye, and DTPA-Gd as the a paramagnetic metal chelate. This imaging agent may be referred to as PG-DTPA-Gd-NIR813 and has the following structure:

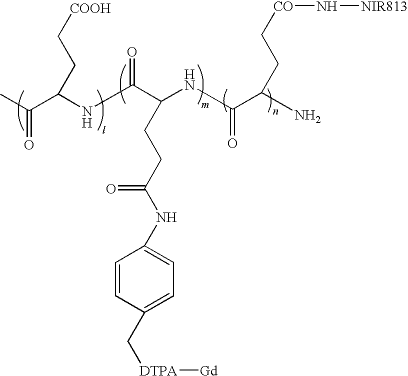

The NIR813 may be present at about <4% w/w linked to PG, for example about 1% w/w linked to PG. The loading of NIR813 should generally be sufficient to minimize any quenching effect.

Another example of an imaging agent comprises poly(L-glutamic acid), IR783 as the NIRF dye, and benzDTPA-Gd as the a paramagnetic metal chelate. This imaging agent may be referred to as PG-DTPA-Gd-NIR783 and has the following structure:

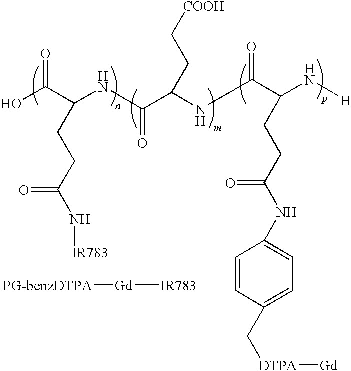

The present disclosure also provides methods for synthesizing NIR813 and imaging agents.

The present disclosure also provides methods for assessing CB activity comprising administering to a subject an imaging agent comprising poly(L-glutamic acid) and a NIRF dye and measuring a NIRF signal.

The present disclosure also provides methods for detecting inhibition of CB activity comprising providing to a plurality of cells an imaging agent comprising poly(L-glutamic acid) and a NIRF dye and a cell and measuring a NIRF signal.

In one example, PG-NIR813 containing 5%-10% of NIR813 may be activated by CB and produce an NIRF signal. The NIRF signal may then be imaged noninvasively and/or measured in a biological sample (e.g., blood) in vitro.

Tumors are known to secrete cathepsin B and/or to contain membrane-associated CB, which is thought to be involved in invasion and metastasis. Therefore, extracellular CB may be used as a target for tumor detection in certain embodiments of the present disclosure. Patients with higher content or increased proteolytic activities of CB in tissue homogenates have significantly higher risk of recurrence or death than the cases with low content of the enzyme. Therefore, CB activity also may be used as aprognostic marker for cancer patients in certain embodiments of the present disclosure. Other diseases that are known to have abnormal activity of CB include atherosclerosis and arthritis. Therefore, imaging agents of the present disclosure that can be used for the assessment of CB activity in cancer may also be used for other diseases.

The present disclosure also provides methods comprising providing to a plurality of cells an imaging agent comprising poly(L-glutamic acid), a NIRF dye, and a paramagnetic metal chelate; and imaging the cells to detect the imaging agent. The imaging agent may be detected with optical or MR imaging or both. When used clinically, such methods may be minimally invasive and offer real-time assessment of anatomic information. Such methods may be used, for example, for SLN mapping.

SLN mapping is used routinely in the clinics using radiolabeled sulfur colloid. Imaging agents that avoid the use of radioisotope and provide the opportunity for SLN imaging using high resolution MRI and high sensitivity optical imaging are advantageous. For example, to prepare one example contrast agent, poly(L-glutamic acid) (PG) was conjugated with paramagnetic metal chelate DTPA-Gd and a fluorescence dye NIR813 to obtain PG-DTPA-Gd-NIR813 conjugate. PG-DTPA-Gd-NIR813 can be used to detect SLN using both optical and MR imaging. The dose required is as low as 0.002 mmol/kg, about 100-fold lower than the clinical dose of Magnevist.

MR and NIRF images were taken before and after subcutaneous injection of PG-DTPA-Gd-NIR813 into the front paw of healthy nude mice or interstitial injection of PG-DTPA-Gd-NIR813 in the tongue of nude mice bearing human DM14 squamous cell carcinoma. After subcutaneous injection, PG-DTPA-Gd-NIR813 colocalized with isosulfan blue dye in the axiliary and branchial lymph nodes, indicating drainage of the contrast agent to the SLN. These nodes were clearly visualized with both T1-weighted MR imaging and NIRF optical imaging within 5 min of contrast injection at a dose of 0.02 mmol Gd/kg (4.8 nmol eq. NIR813), while the branchial nodes were more readily detected with NIRF imaging than with MRI at a lower dose of 0.002 mmol Gd/kg (48 nmol eq. NIR813). In the head and neck area after interstitial injection of PG-DTPA-Gd-NIR813 into the tongue (15 μL, 0.02 mmol Gd/kg), optical imaging identified all 6 cervical nodes in tumor bearing mice. In comparison, 4 of the 6 nodes were detected by MRI, and contrast enhancement of these nodes were reduced compared to nodes in healthy mice. Histopathologic examinations of sentinel nodes resected under NIRF imaging guidance revealed the presence of micrometastases in 4 of 6 nodes. The superior spatial resolution of MRI combined with high detection sensitivity of NIRF imaging enabled preoperative visualization of sentinel nodes with accurate anatomic location and detection of abnormal contrast enhancement, while intraoperative NIRF imaging permitted selective removal of SLN and subsequent identification of micrometastases in these nodes. This example method represents a minimally invasive approach toward lymph node mapping with sentinel node biopsy.

PG-DTPA-Gd-NIR813 is a polymeric contrast agent having hydrodynamic volume of greater than 20 nm. In general, the size of lymphangiographic agents for SLN mapping may be large enough to avoid their leakage into the blood capillaries and rapid loss of signal, but small enough to remain mobile for rapid transit within the lymphatic tract. Contrast agents having hydrodynamic diameter 5-40 nm usually satisfy this criterion. Example agents may be derived using the present disclosure and Kim S, Lim Y T, Soltesz E G, et al. Near-infrared fluorescent type II quantum dots for sentinel lymph node mapping. Nat Biotechnol 2004; 22:93-97; Moghimi S M. Bonnemain B. Subcutaneous and intravenous delivery of diagnostic agents to the lymphatic system: applications in lymphoscintigraphy and indirect lymphography. Adv Drug Deliv Rev 1999; 37:295-312. In addition to a suitable size, it may also be desirable to obtain a biocompatible contrast agent that can be metabolized and eventually cleared from the body. The polymeric carrier in PG-DTPA-Gd-NIR813 is a biodegradable polymer, which has demonstrated excellent biocompatibility. In various studies in rodents, PG was used at doses from 200 to 800 mg/kg without causing apparent toxic effects after intravenous injection. Li C. Poly(L-glutamic acid)—anticancer drug conjugates. Adv Drug Deliv Rev 2002; 54:695-713. In fact, polymeric anticancer agents based on PG have advanced into clinic trial studies. Because a large fraction of PG-DTPA-Gd-NIR813 injected interstitially would eventually be removed by lymph nodes with little to none of the contrast agent entering systemic circulation, this agent may have acceptable toxicity profile in SLN mapping at doses that are 10- to 100-fold less than the dose of conventional MRI contrast agent used clinically for intravenous injection.

In yet other embodiments the imaging agents may further comprise a therapeutic agent. These imaging agents may be referred to as biodegradable drug carriers. One example of such imaging agents may comprise a therapeutic agent, poly(L-glutamic acid), and a NIRF dye.

Biodegradable drug carriers may be used to monitor the delivery of therapeutic agents. Accordingly, the present disclosure provides, in certain embodiments, methods for imaging degradation of polymeric drug carriers comprising introducing to a cell a polymeric drug carrier comprising a therapeutic agent, poly(L-glutamic acid), and a NIRF dye; and imaging the cell using near-infrared fluorescence imaging.

Recently, MRI agents consisting of dendrimers have been developed for preoperative characterization of lymphatic drainage and lymph node metastases from mammary tumors. Kobayashi H, Kawamoto S, Sakai Y, et al. Lymphatic drainage imaging of breast cancer in mice by micro-magnetic resonance lymphangiography using a nano-size paramagnetic contrast agent. J Natl Cancer Inst 2004; 96:703-708; Kobayashi H, Kawamoto S, Bernardo M, et al. Delivery of gadolinium-labeled nanoparticles to the sentinel lymph node: comparison of the sentinel node visualization and estimations of intra-nodal gadolinium concentration by the magnetic resonance imaging. J Control Release 2006; 111:343-351. These studies demonstrated that the superior temporal and spatial resolution of micro-MR imaging facilitates the identification of lymphatic metastasis in experimental animals.

In embodiments of the present disclosure, using a dual modality contrast agent in mice with lymph node metastases from squamous carcinoma tumor implanted in the tongue, T1-weight MR images confirmed that preoperative MRI may allow for differentiation of normal and metastatic nodes. The different pattern in lymph node enhancement may result from differences in macrophage uptake of macromolecular contrast agents between normal and metastatic lymph nodes, as has been shown to be the case for superparamagnetic iron oxide nanoparticles. Anzai Y. Prince MR. Iron oxide-enhanced MR lymphography: the evaluation of cervical lymph node metastases in head and neck cancer. J Magn Reson Imaging 1997; 7:75-81; Anzai Y, Blackwell K E, Hirschowitz S L, et al. Initial clinical experience with dextran-coated superparamagnetic iron oxide for detection of lymph node metastases in patients with head and neck cancer. Radiology 1994; 192:709-715; Harisinghani M G, Barentsz J, Hahn P F, et al. Noninvasive detection of clinically occult lymph-node metastases in prostate cancer. N Engl J Med 2003; 348:2491-2499.

Although MRI is a useful method for precise localization and preoperative characterization for the presence or absence of metastases in SLN, NIRF imaging allows detection of SLN at a much higher sensitivity. At an injected dose of 0.02 mmol Gd/kg, one may detect the same sets of SLN as soon as 3 min after the injection of PG-DTPA-Gd-NIR813 with both MRI and optical imaging. However, at a reduced dose of 0.002 mmol Gd/kg, MRI detected only one of the two lymph nodes that were visualized with NIRF imaging. Moreover, while NIRF imaging was able to detect all 6 cervical lymph nodes containing micrometastases in mice with squamous carcinoma tumor in the tongue, MRI revealed enhancement in 4 of the 6 nodes. These findings are consistent with lower detection sensitivity with MRI than with NIRF imaging.

The challenge for implementation of sentinel lymph node biopsy is to develop a reliable minimally invasive technique with high resolution and high sensitivity. Embodiments of the present disclosure relate to a dual-functional magnetic resonance (MR) and optical, such as near-infrared fluorescence (NIRF) optical imaging contrast agent. This agent may, in certain embodiments, be used for both preoperative and intraoperative sentinel node detection.

In more specific embodiments, the NIRF imaging agent may include a near infrared fluorophore, such as a near infrared dye. The near infrared dye may include a cyanine or indocyanine derivative such as Cy5.5. The MRI agent may include Gd, Mn or iron oxide.

Dual MRI and optical imaging of with PG-DTPA-Gd-NIR813 may be of value for the detection of SLN. NIRF eliminates the need for both a radioactive tracer and a blue dye. Kim et al. have shown that lymph flow and the SLN can be identified optically and in real time, using intraoperative NIRF imaging and QD. Kim S, Lim Y T, Soltesz E G, et al. Near-infrared fluorescent type II quantum dots for sentinel lymph node mapping. Nat Biotechnol 2004; 22:93-97.

One example dual modality imaging technique may be used in the following clinical scenario. Initially, MRI may be used for noninvasive detection of lymph node metastases. If the presence of lymph node metastases is confirmed nonequivocally with MRI alone, surgery to remove the whole nodal basin may be performed, thus eliminating the SLN biopsy step and the associated waiting period. If MRI is unable to detect metastases with a high degree of certainty, SLN mapping and subsequent SLN biopsy may then be performed using NIRF imaging. This may permit intraoperative dissection without the use of ionizing radiotracer. Because of its high detection sensitivity, NIRF imaging may also be used to inspect the surgical site to ensure complete removal the SLN.

Accordingly dual functional macromolecular contrast agents according to embodiments of the present disclosure may be suitable for both MR and NIRF optical imaging. Such an agent may be useful not only for precise localization of SLN and preoperative characterization of lymph node abnormalities using MRI, but also for the SLN mapping and monitoring the success of complete resection of SLN during surgical operation.

To facilitate a better understanding of the present invention, the following examples of specific embodiments are given. In no way should the following examples be read to limit or define the entire scope of the invention.

### EXAMPLES

### Example 1

### PG-NIR813

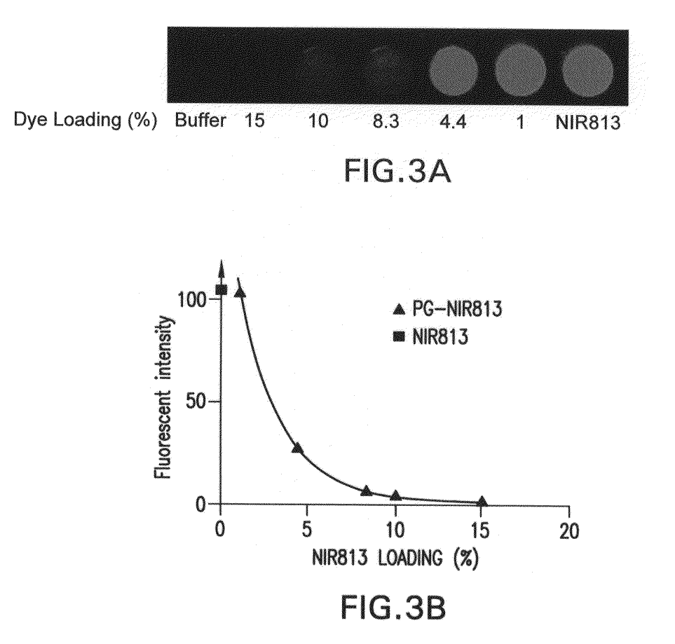

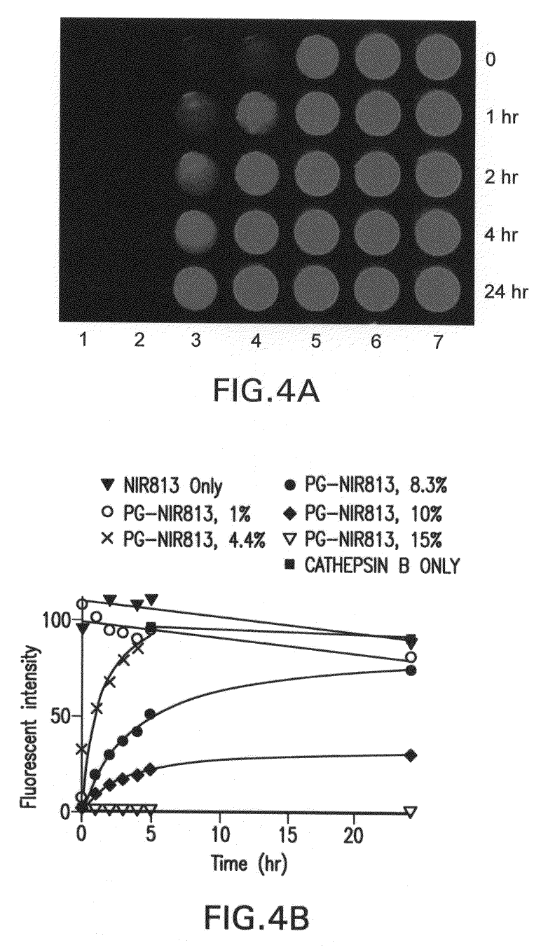

Near-infrared fluorescence signal in PG-NIR813 is efficiently quenched when NIR813 loading is greater than about 4% (based on the number of repeating glutamic acid units in the PG polymer) as shown in FIG. 3. However, when the loading is greater than about 15%, polymer cannot be degraded by CB, as shown in FIG. 4. Therefore, in some examples, the optimal loading for certain activatable NIRF probe may be between about 4% and about 15%.

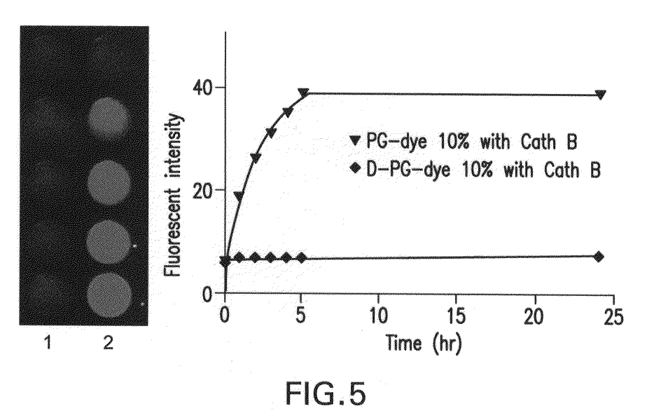

As shown in FIG. 5, D-PG-NIR813 is not degradable by CB. Therefore, D-PG conjugated dye may be used as carrier for the design of activatable NIRF probe responsive to other enzymes such as MMP-2. In such design, the NIRF fluorophore (NIR813 or others) may be attached to the side chains of D-PG through peptide linkers that are specific substrate for the enzymes of interest.

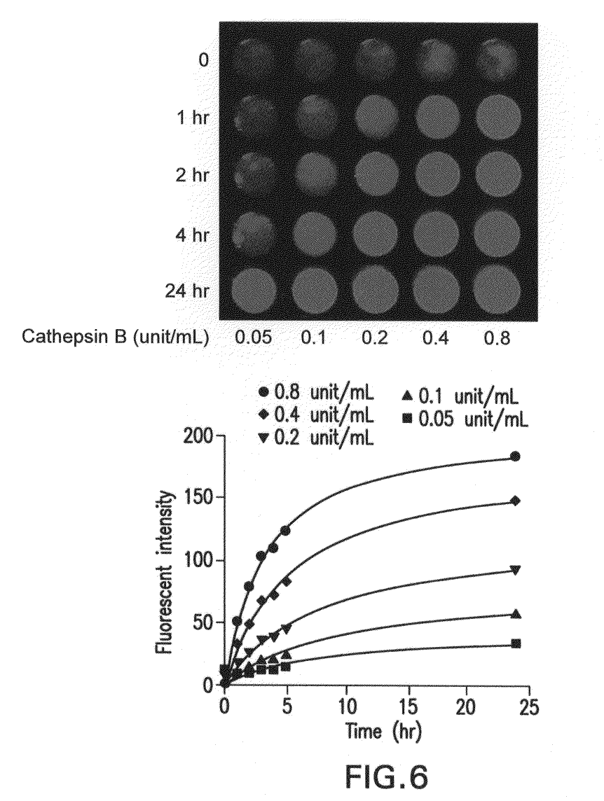

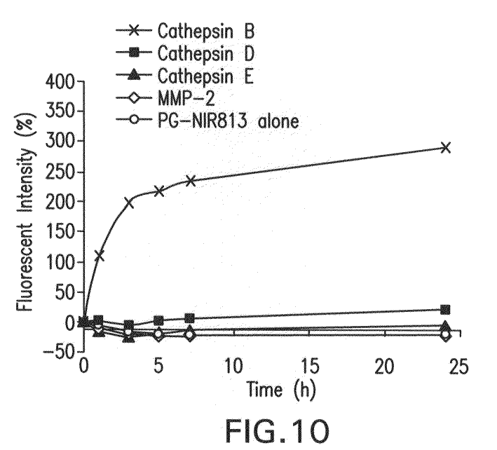

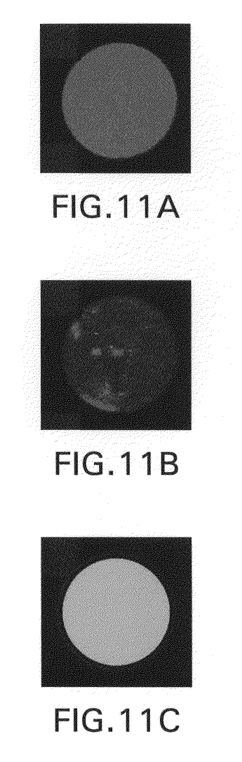

As shown in FIG. 6, PG-NIR813 is degraded by CB in a dose-dependent manner. PG-NIR813 is not degraded by other proteinases tested (FIG. 10). Thus, PG-NIR813 may be used to quantify CB activity in biological fluids (such as plasma) in in vitro settings.

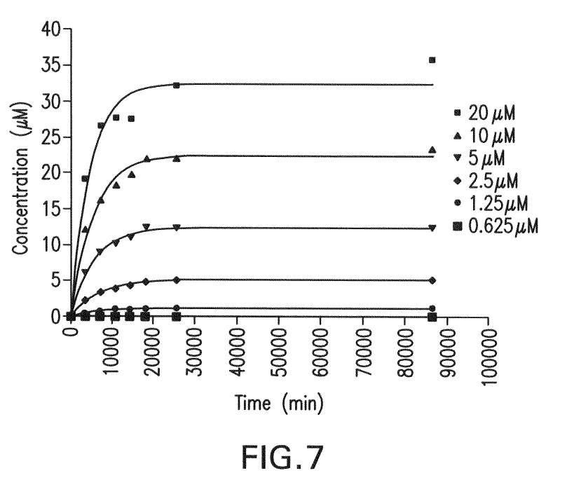

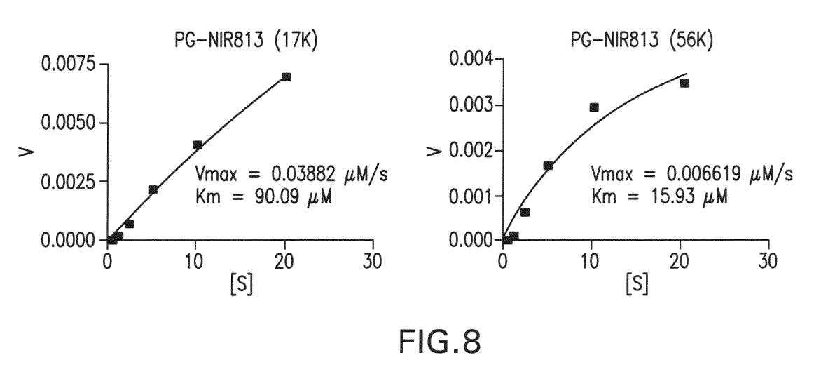

The degradation of PG-NIR813 conjugate is generally a function of polymer molecular weight. Conjugates with higher molecular weight degrade at a slower rate, as shown in FIG. 7 and FIG. 8.

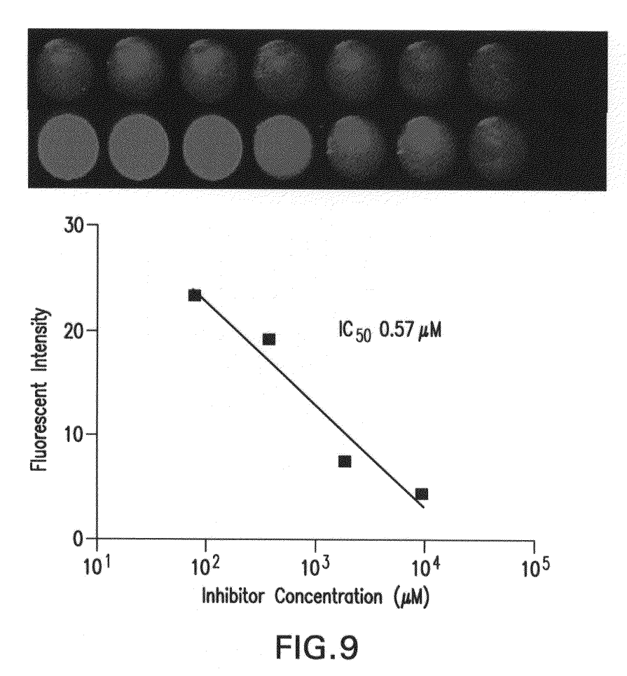

As shown in FIG. 9, degradation of PG-NIR813 by CB can be inhibited by CB inhibitor in a dose-dependent manner. Accordingly, this property may be used to screen for CB inhibitors in a high-throughput setting. PG-NIR813 may also be used to image the inhibition of CB activity by CB inhibitors in vivo.

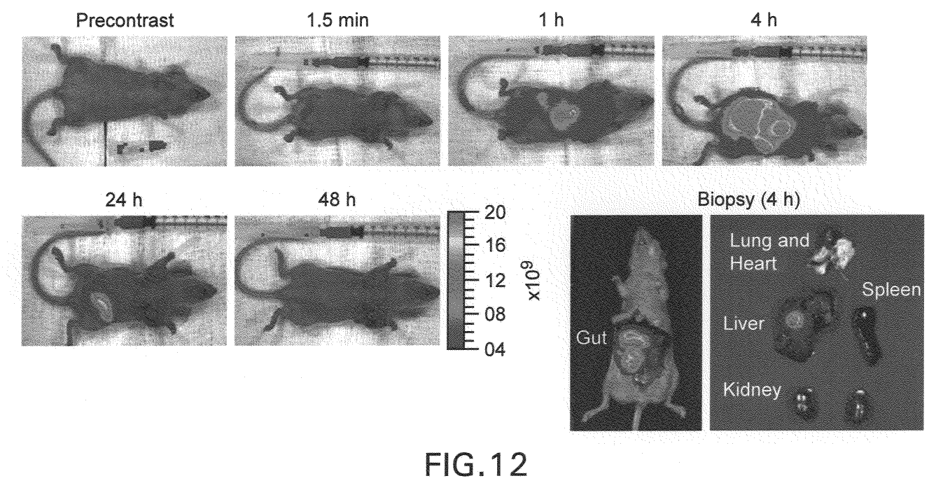

As shown in FIG. 12, PG-NIR813 degradation in vivo can be monitored noninvasively. Considering the structural similarity between PG-NIR813 and PG-paclitaxel that is in advanced clinical trial studies, PG-NIR813 may be used to select patients who may benefit the most from PG-paclitaxel therapy, because the efficacy of PG-paclitaxel is dependent on the degradation of and release of paclitaxel at the target site.

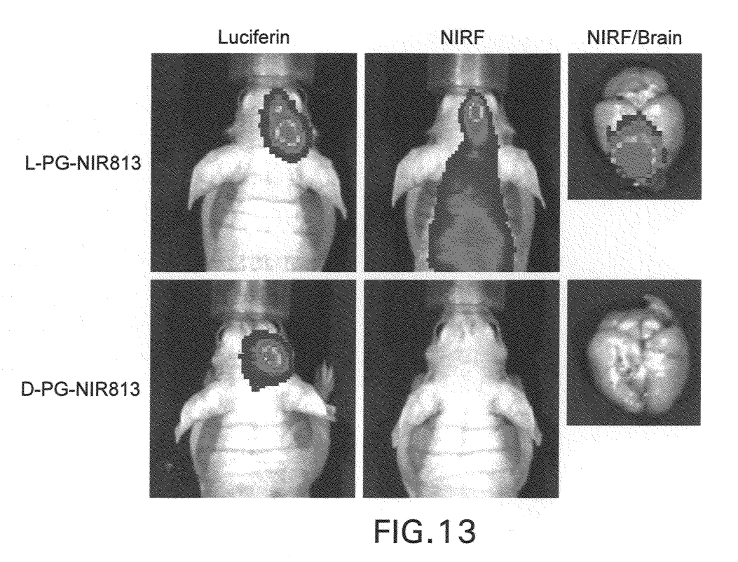

As shown in FIG. 13, PG-NIR813 can be used to detect the CB activity in vivo.

### PG-DTPA-Gd-NIR813

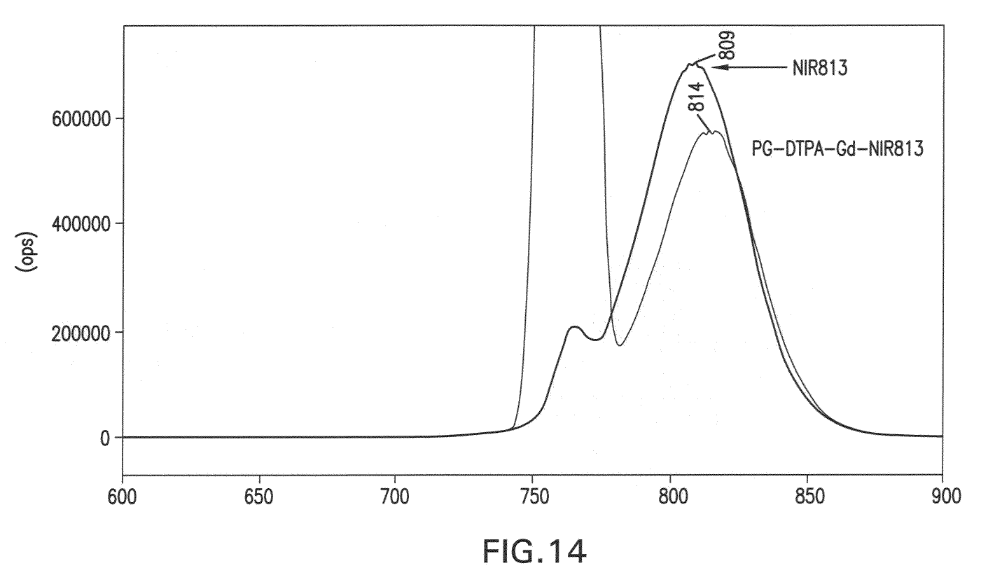

Poly(L-glutamic acid) (PG) was conjugated with paramagnetic metal chelate Gd-DTPA and a fluorescence dye NIR813 to obtain PG-DTPA-Gd-NIR813 conjugate. The fluorescence spectrum is shown in FIG. 14.

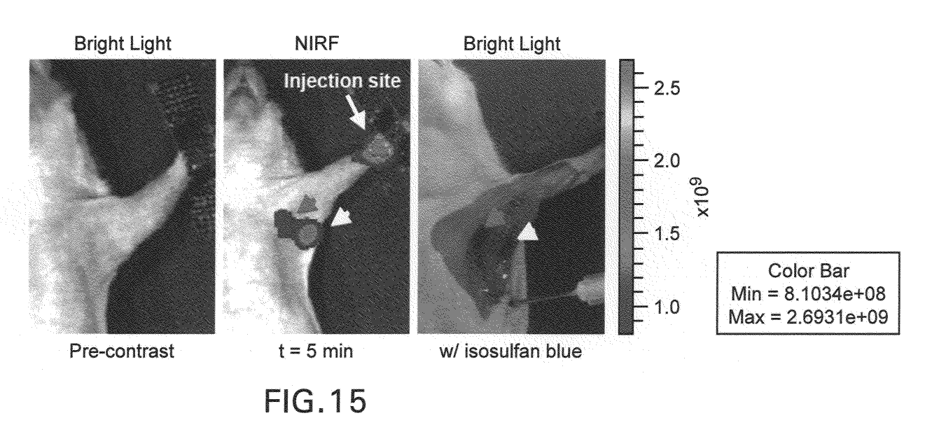

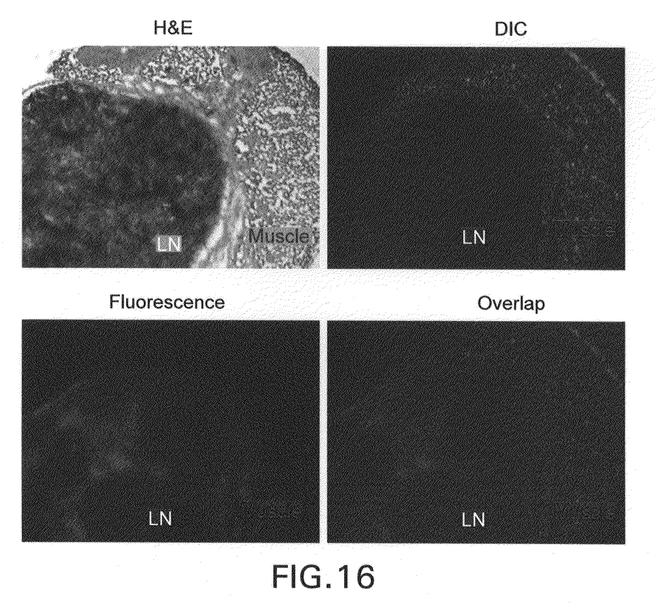

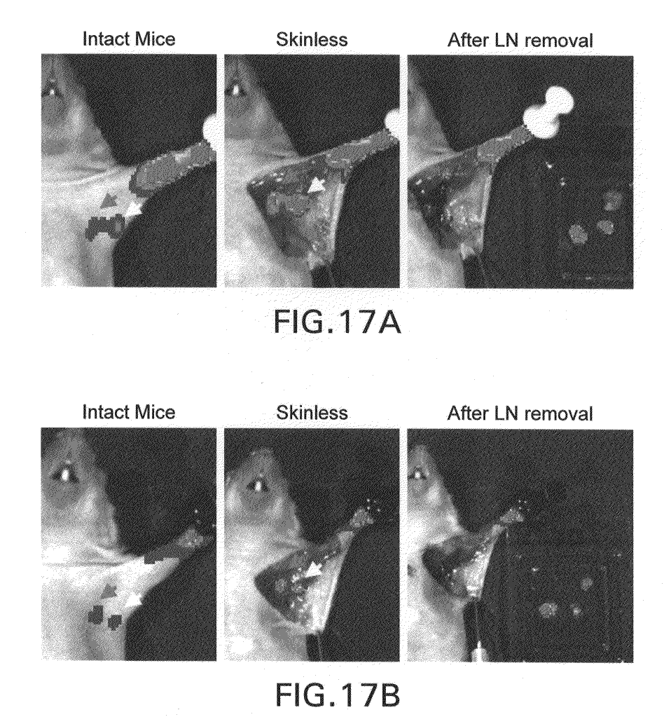

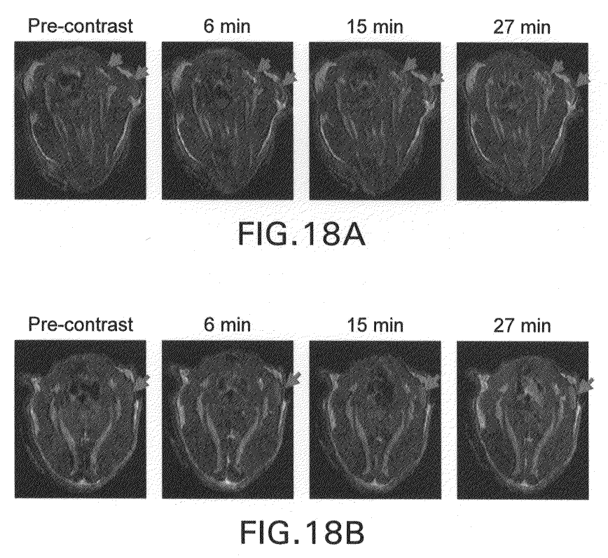

To determine its localization in the SLN, PG-DTPA-Gd-NIR813 was co-injected with isosulfan blue dye, the gold standard for SLN mapping. Pre- and post-contrast images were taken using 4.7T Bruker Biospec MRI scanner and Xenogen optical imaging system. PG-DTPA-Gd-NIR813 was injected subcutaneously into the front paw of nude mice at doses ranging from 0.002 mmol Gd/kg (4.8 nmol eq. NIR813) to 0.02 mmol Gd/kg (48 nmol eq. NIR813). When injected together with isosulfan blue dye, PG-DTPA-Gd-NIR813 co-localized with isosulfan blue dye, indicating drainage of the contrast agent to the SLN (FIG. 15). Axiliary and branchial lymph nodes did not have sufficient contrast with neighboring tissue to be identified without contrast in T-1 weighted acquisitions (FIG. 14). However, these nodes were clearly visualized as soon as 3 min with both MR and optical imaging within 6 min of contrast injection, even at the lowest dose tested (0.002 mmol Gd/kg) (FIG. 15 and FIG. 16). Enhancement remained persistent beyond 24 hr after injection (FIG. 16).

The superior spatial resolution of MRI combined with high detection sensitivity with NIR optical imaging enabled visualization of lymphatic flow and SLN using a minimally invasive imaging procedure requiring no ionizing radiation, and may provide a powerful method for SLN mapping.

### Example 2

### Materials & Methods

The following materials and methods were used to create the agents in this example

Poly(L-glutamic acid) sodium salt, 1,3-diisopropylcarbodiimide (DIC), pyridine, N-hydroxysuccinamide (NHS), N,N-diisopropylethylamine (DIPEA), IR-783 sodium acetate (NaOAc), EDTA, cysteine, PBS (0.01 M phosphate-buffered saline containing 138 mM NaCl and 2.7 mM KCl, pH 7.4), N,N′-dimethylaminopyridine (DMAP), and CB were purchased from Sigma-Aldrich (St. Louis, Mo.). 1-Hydroxybenzotriazole (HOBt), benzotriazol-1-yl-oxy-tris-pyrrolidino-phosphonium hexafluorophosphate (PyBOP), and N-tert-butoxycarbonyl-1,5-diaminopentane toluenesulfonic acid salt was purchased from Novabiochem (San Diego, Calif.). Trifluoroacetic acid (TFA) was obtained from Chem-Impex International, Inc. (Wood Dale, Ill.). 4-Mercaptobenzoic acid was purchased from TCI (Portland, Oreg.). Spectra/Pro 7 dialysis tubing with molecular weight cutoff (MWCO) of 10 000 was purchased from Fisher Scientific (Pittsburgh, Pa.). PD-10 columns came from Amersham-Pharmacia Biotech (Piscataway, N.J.). CB inhibitor Ac-LVK-CHO (Inhibitor II) was purchased from Calbiochem (La Jolla, Calif.). All solvents were purchased from VWR (San Dimas, Calif.).

Analytical Methods

Analytical high-performance liquid chromatography (HPLC) was carried out on an Agilent 1100 system (Wilmington, Del.) equipped with a Vydac peptide and protein analytic C-18 column (Anaheim, Calif.). Sample was eluted with H₂O and acetonitrile containing 0.1% TFA varying from 10% to 80% over 30 min. Fluorescence intensity was measured by Licor Odyssey instrument (Lincoln, Nebr.).

Synthesis of IR-783-S-Ph-COOH

IR-783 (250 mg, 0.33 mmol) and 4-mercaptobenzoic acid (104 mg, 0.67 mmol) were dissolved in 5 mL DMF and stirred for overnight at room temperature. After removing the solvent, the residue was dissolved in methanol and precipitated in ether. The solid was collected by filtration and further purified with flash chromatography using ethyl acetate and methanol as the mobile phase.

Synthesis of IR-783-S-Ph-CONH(CH₂)₅NHBoc

IR-783-S-Ph-COOH (150 mg, 0.18 mmol), NHS (22 mg, 0.21 mmol) and were dissolved in 5 mL DMF. DIC (31 μL, 0.21 mmol) and DMAP (2.5 mg, 0.02 mmol) were added to the solution. The mixture was stirred at room temperature for 4 hr. The solvents were removed under vacuum. The residue was washed with ether. The resulting activated ester IR-783-S-Ph-CO—NHS and BocNH(CH₂)₅NH₂ (42 mg, 0.21 mmol) were dissolved in 5 mL DMF with 5% DIPEA. The mixture was stirred for 4 hr. After removing the solvent, the residue was dissolved in methanol and precipitated in ether. The solid was filtered out and further purified with flash chromatography with ethyl acetate and methanol.

Synthesis of IR-783-S-Ph-CONH(CH₂)₅NH₂ (NIR813)

IR-783-S-Ph-CONH(CH₂)₅NHBoc was dissolved in 20 mL of 40% TFA in di chloromethane and stirred for 25 min. The solvent was removed under vacuum. The residue was dissolved in methanol and precipitated in ether. The solid was filtered out and then dissolved in acetonitrile and water. The product was dried by lyophilization. MS: 929.47 (calcl.), 929.43 (found, M⁺).

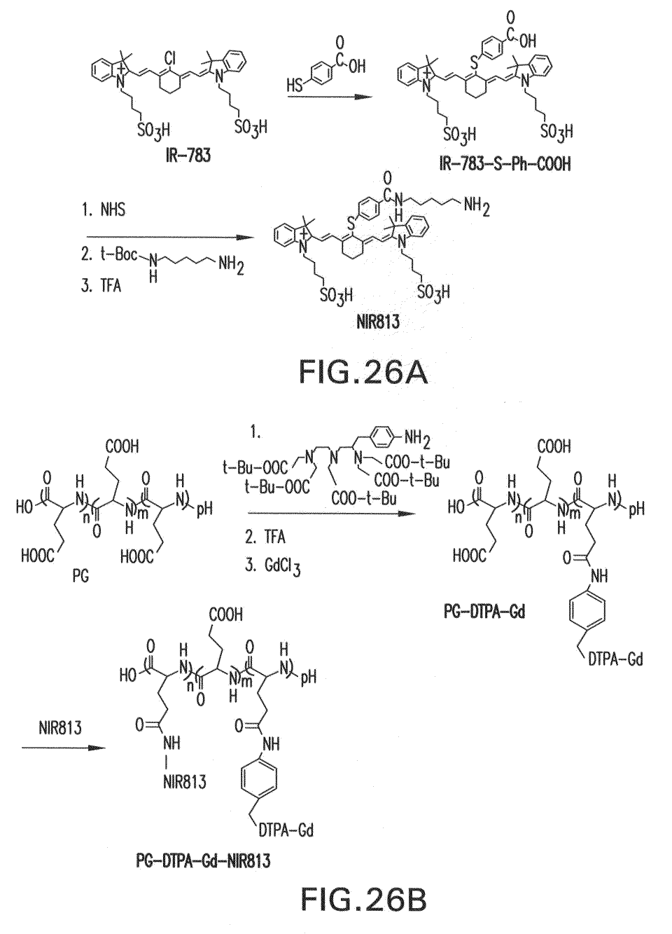

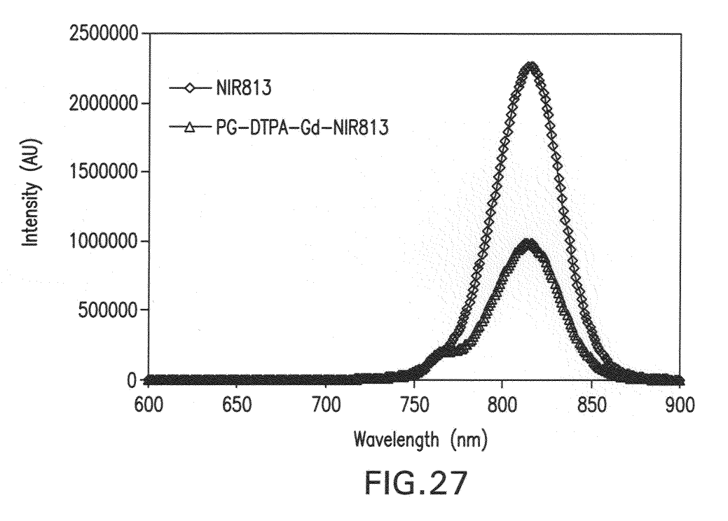

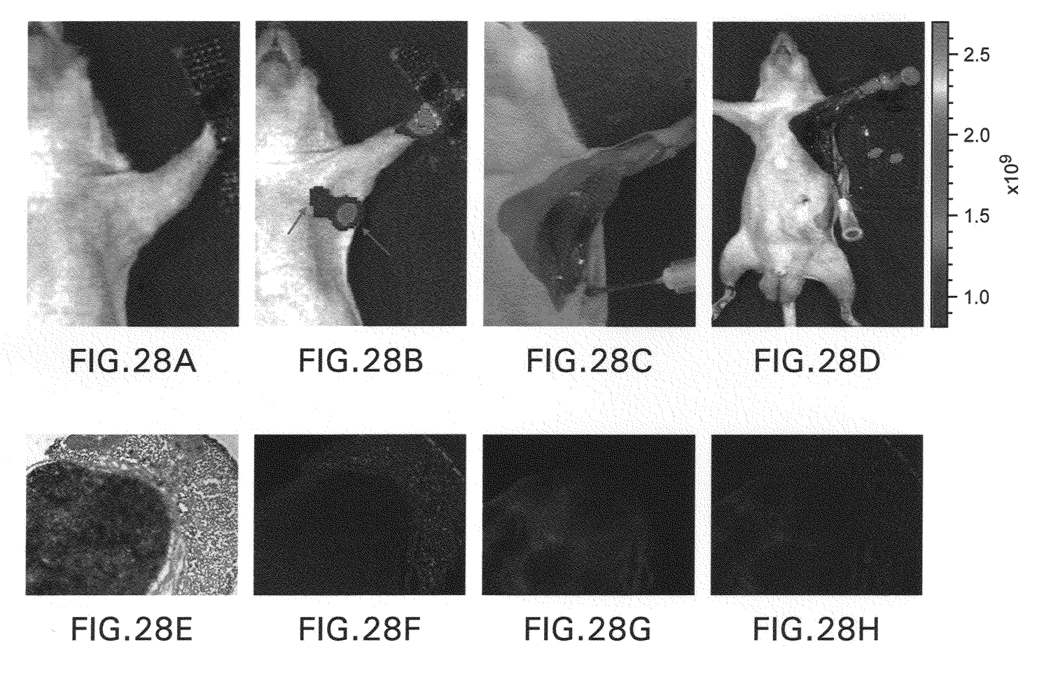

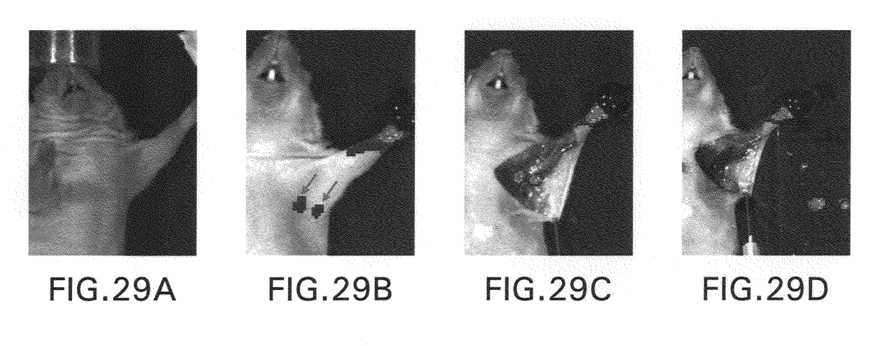

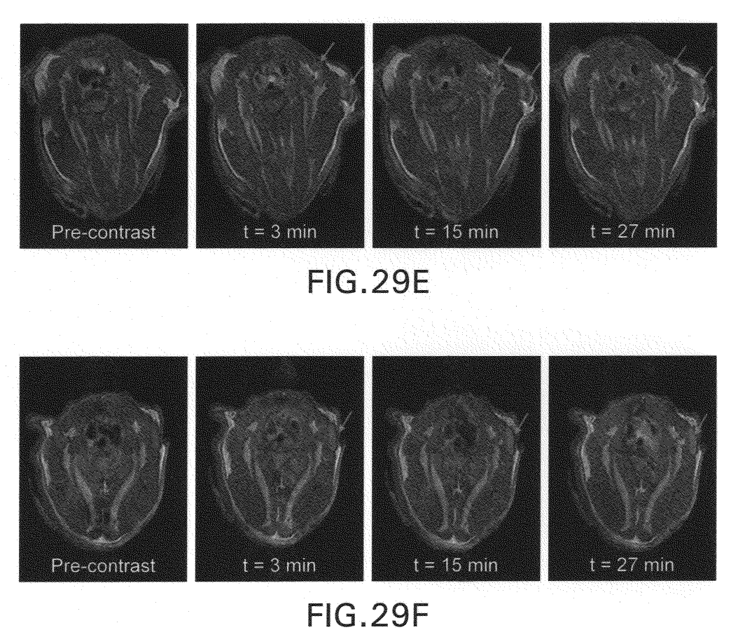

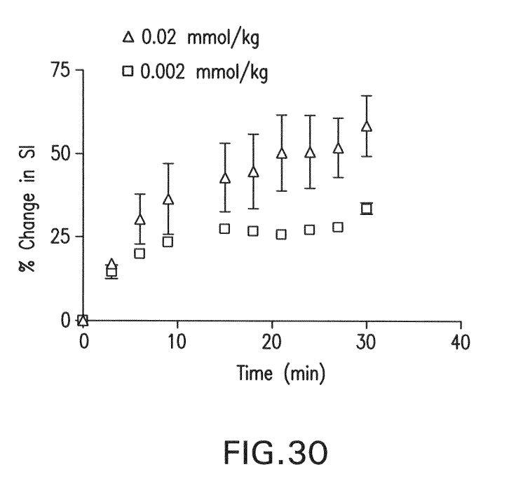

NIRF dye containing a primary amine, IR-783-S-Ph-CONH(CH₂)₅NH₂, was synthesized in 3 steps (FIG. 26A). IR-783-S-Ph-COOH was first synthesized according to Strekowski et al. Strekowski L, Gorecki T, Mason J C, Lee H. Patonay G. New Heptamethine Cyanine Reagents for Labeling of Biomolecules with a Near-Infrared Chromophore. Heterocyclic communications 2001; 7:2 117-2122. Briefly, IR-783 (250 mg, 0.33 mmol) and 4-mercaptobenzoic acid were dissolved in 5 mL dimethylformamide (DMF). This solution was stirred overnight at room temperature. After removing the solvent, the residue was dissolved in methanol and precipitated in ether. The solid was collected by filtration and further purified with flash chromatography using ethyl acetate and methanol as the mobile phase. IR-783-S-Ph-COOH was then conjugated to t-Boc protected heterodiamine t-BocNH(CH₂)₅NH₂ using activated ester. Thus, IR-783-S-Ph-COOH (150 mg, 0.18 mmol) and NHS (22 mg, 0.21 mmol) were dissolved in 5 mL DMF together with 1,3-diisopropylcarbodiimide (31 μL, 0.21 mmol) and 4-dimethylaminopyridine (2.5 mg, 0.02 mmol). The reaction proceeded at room temperature for 4 hr, after which the solvent was removed under vacuum and the residue washed with ether. The resulting IR-783-S-Ph-CO—NHS was reacted with BocNH(CH₂)₅NH₂ (42 mg, 0.21 mmol) for 4 hr in 5 mL DMF containing 5% N,N-diisopropylethylamine. The product was then worked up and purified with flash chromatography. Finally, the t-Boc protection group in IR-783-S-Ph-CONH(CH₂)₅NHBoc was removed by treating with 40% TFA in dichloromethane. After solvent removal, the product was purified by precipitation from a methanol solution with ether. IR-783-S-Ph-CONH(CH₂)₅NH₂, was collected by filtration and dried by lyophilization. MS: 929.47 (calcl.), 929.43 (found, M⁺). The fluorescence emission maximum for IR-783-S-Ph-CONH(CH₂)₅NH₂ was 813 nm (FIG. 27). Consequently, IR-783-S-Ph-CONH(CH₂)₅NH₂ is termed NIR813 dye throughout this disclosure.

Synthesis of PG-NIR813

Sodium salt of poly-L-glutamic acid (number-average molecular weight Mₙ, 17,500 and 56,000) was dissolved in H₂O and precipitated by acidifying with 1 N HCl. The polymer precipitate was collected by centrifugation and dried by lyophilization. The percentage of dye used for each loading was based on molar number of the side chain glutamic acid residues in pre-weighted PG. The amounts of PyBOP and HOBt were 2 eq of the NIR813 dye. All of the reactants PG, NIR813, PyBOP and HOBt were dissolved in DMF. 2% of DIPEA was added to the solution. The mixture was stirred until the dye peak disappeared on HPLC (about 2 to 4 hr). The solvents were removed under vacuum. The residue was dissolved in PBS and purified using PD-10 columns eluted with PBS. The solution was dialyzed against H₂O overnight and lyophilized. The yields of polymer were around 60%.

To determine the dynamic range of NIR813 dye, a stock solution of 200 μM of NIR813 in methanol was diluted with assay buffer (20 mM of NaOAc, 1 mM EDTA, 5 mM cysteine, pH 5.0) to 2.5, 5, 10, 15, 20 μM solutions. 100 μl, of each sample was put in each well. The fluorescence intensity for each concentration was collected by Licor Odyssey camera. The result was reported by the plot of concentration vs. fluorescence intensity.

Quenching Effect and Stability Test of PG-NIR813 with Different Loading (1%, 4.4%, 8.3%, 10% and 15%)

L-PG-NIR813 with different loading (1%, 4.4%, 8.3%, 10% and 15%) was dissolved in assay buffer respectively to form 10 μM solutions. 100 μL of each sample was put in each well. The fluorescence intensity of each sample was determined using Li-cor Odyssey NIRF imager. The result of quenching effect was showed in the plots of loading percentage vs. fluorescence intensity. The microwell assay plate was incubated at 37° C. for 48 hr. At predetermined time intervals, the stability of each sample in each well was checked through the change on fluorescence intensity. The stability of each loading was indicated by the plots of time vs. fluorescence intensity.

Biodegradation of L-PG-NIR813 with Different Loading (1%, 4.4%, 8.3%, 10% and 15%)

L-PG-NIR813 with different loading (1%, 4.4%, 8.3%, 10% and 15%) and CB were dissolved in assay buffer respectively. The reaction mixture in each well (100 μL) was composed of 10 μM L-PG-NIR813 probe and 0.4 units/mL CB. The samples were incubated at 37° C. for 24 hr. At predetermined time intervals, the fluorescence intensity of reaction mixture in each well was measured using Li-Cor Odyssey imager. The result of each sample was showed in the plots of time vs. fluorescence intensity.

Concentration Effects on Biodegradation of L-PG-NIR813 with Loading 8.3% and 10%.

L-PG-NIR813 with different loading 8.3% and 10% and CB were dissolved in assay buffer respectively. Three different concentrations 5, 10, and 20 μM were prepared for each loading of L-PG-NIR813. The concentrations of CB were serially arranged from 0.05 to 0.8 units/mL for each concentration of the probe. The total volume in each well was 100 μL. The reaction mixtures were incubated at 37° C. for 24 hr. At predetermined time intervals, the fluorescence intensity of reaction mixture in each well was measured by Li-Cor Odyssey imager. The result was showed in the plots of time vs. fluorescence intensity.

Inhibition of Biodegradation of L-PG-NIR813 in Presence of CB Inhibitor II

L-PG-NIR813 with different loading 8.3% and 10%, CB and CB inhibitor II were dissolved in assay buffer respectively. The reaction mixture (100 μL) in each well was composed of 10 μM L-PG-NIR813 probe and 0.2 units/mL CB. The CB inhibitor II was serially diluted in the assay buffer to obtain concentrations ranging from 240 μM to 77 nM. The microwell assay plate was incubated at 37° C. for 24 hr. At predetermined time intervals, the inhibition of biodegradation was examined using Li-Cor Odyssey imager. The result was showed in the plots of time vs. fluorescence intensity.

Physicochemical Properties of Peptidyl MMP-2 Inhibitors are shown in Table 1.

TABLE 1

| | | | | HPLC |
|---|---|---|---|---|
| | Mass Spectrometry | | | Retention |
| | Molecular | Calculated | Observed | Time |
| Dye | formula | MW | (M + 1) | (min)^{a} |
| IR-783-S-Ph-COOH | C₄₅H₅₃N₂O₈S₃⁺ | 845.31 | 845.37 | 18.59 |
| IR-783-S-Ph- | C₅₀H₆₅N₄O₇S₃⁺ | 929.47 | 929.43 | 17.88 |
| CONH(CH₂)₅NH₂ (NIR813) | | | | |

^{a}Sample was eluted with H₂O and acetonitrile containing 0.1% TFA varying from 10% to 80% over 30 min.

### Example 3

### Materials and Methods

The following materials and methods were used to create the agents in this example.

PG sodium salt; 1,3-diisopropylcarbodiimide (DIC); pyridine; 4-dimethylaminopyridine (DMAP); trifluoroacetic acid (TFA); gadolinium (III) chloride hexahydrate; PBS (0.01 M phosphate buffered saline (PBS) containing 138 mM NaCl and 2.7 mM KCl, pH 7.4); 1-ethyl-3-(3-dimethylaminopropyl)-carbodiimide (EDC); 2-morpholinoethanesulfonic acid buffer (MES); IR-783 dye; N-hydroxysuccinimide (NHS); N,N-diisopropylethylamine (DIPEA); isosulfan blue; and all the other reagents and solvents were purchased from Sigma-Aldrich (St. Louis, Mo.). N-tert-butoxycarbonyl-1,5-diaminopentane toluenesulfonic acid salt was purchased from Novabiochem (San Diego, Calif.). 4-mercaptobenzoic acid was purchased from TCI (Portland, Oreg.). P-aminobenzyl-diethylenetriaminepenta (acetic acid-t-butyl ester) was obtained from Macrocyclics (Dallas, Tex.). Spectra/Pro 7 dialysis tubing with molecular weight cutoff (MWCO) of 10,000 and PD-10 columns came from Amersham-Pharmacia Biotech (Piscataway, N.J.).

Analytical Methods

Gel permeation chromatography (GPC) was performed on a Waters (Milford, Mass.) high-performance liquid chromatography (HPLC) system consisting of a 600 controller, a 717 plus auto sampler, and a Viscotek E-Z^{pro} triple detector (Viscotek, Houston, Tex.) that records refractive index, viscosity, and light-scattering signals. The samples were separated using an TSK-G4000PW 4.6 mm×30 cm column (TosoHaas, Montgomeryville, Pa.) eluted with PBS containing 0.1% LiBr at a flow rate of 1.0 ml/min. Number-average molecular weights of the polymer conjugates were calculated using Viscotek TriSEC GPC software. Elemental analysis was performed by Galbraith Laboratories, Inc. (Knoxyille, Tenn.).

Analytical high-performance liquid chromatography (HPLC) was carried out on an Agilent 1100 system (Wilmington, Del.) equipped with a Vydac peptide and protein analytic C-18 column (Anaheim, Calif.). Sample was eluted with water and acetonitrile containing 0.1% TFA varying from 10% to 80% over 30 min. Fluorescence intensity was measured by Spex Fluorolog spectrofluorimeter (Jobin Yvon Inc, Edison, N.J.).

Synthesis of IR783-NH₂

IR-783 (250 mg, 0.33 mmol) and 4-mercaptobenzoic acid were dissolved in 5 mL DMF. This solution was stirred overnight at room temperature. After removing the solvent, the residue, which is IR-783-S-Ph-COOH, was dissolved in methanol and precipitated in ether. The solid was filtered out and further purified with flash chromatography with ethyl acetate and methanol.

IR-783-S-Ph-COOH (150 mg, 0.18 mmol) and NHS (22 mg, 0.21 mmol) were dissolved in 5 mL DMF. DIC (31 μL, 0.21 mmol) and DMAP (2.5 mg, 0.02 mmol) were added to the solution and the mixture was stirred for 4 hours. The solvents were removed under vacuum and the residue was washed with ether. This gives the green residue, IR-783-S-Ph-COOSu.

IR-783-S-Ph-COOSu was dissolved in 5 mL DMF and was added with BocNH(CH₂)₅NH₂ (42 mg, 0.21 mmol) and 5% DIPEA. The mixture was stirred for 4 hours. After removing the solvent, the residue, which is IR-783-S-Ph-CONH(CH₂)₅NHBoc, was dissolved in methanol and precipitated in ether. The solid was filtered out and further purified with flash chromatography with ethyl acetate and methanol.

The t-Boc protection of IR-783-S-Ph-CONH(CH₂)₅NHBoc was removed by dissolving this residue in 20 mL of 40% TFA in DCM and was stirred for 25 min. The solvent was removed under vacuum and the resulting material was dissolved in methanol and precipitated in ether. The final product, IR783-NH₂, was filtered out and then dissolved in acetonitrile and water. The product was dried by lyophilization and was characterized using NMR and mass spectrometry (MS).

Synthesis of PG-DTPA-Gd

PG (Mₙ, 41,400; 1 g, 7.75 mmoles of carboxylic unit) and p-aminobenzyl-diethylenetriaminepenta(acetic acid-t-butyl ester) (2.1 g, 2.79 mmoles) were dissolved in 10 ml of anhydrous DMF, followed by the addition of 1,3-diisopropylcarbodiimide (403 mg, 3.1 mmoles), 1.2 ml of pyridine, and trace amount of 4-dimethylaminopyridine. The reaction mixture was stirred at 4° C. overnight. To remove the protecting groups, the reaction mixture was treated with TFA at 4° C. overnight. After removal of TFA under vacuum, 20 ml of ice-cold 1M NaHCO₃ was added into the residual solid. The pH of the solution was brought up to 7.5 with 1 M NaOH and the solution was dialyzed against PBS and water sequentially (MWCO 10,000). The resulting solution was filtered through 0.2 μm membrane filters and lyophilized. About 28 of 274 glutamic acid residues were coupled to benzylDTPA-Gd, determined by elemental analysis. Into a PG-Benz-DTPA (110 mg) solution in 10 ml of sodium acetate buffered aqueous solution (0.1 M, pH 5.5) was added 0.37 ml of GdCl₃.6H₂O (100 mg/ml, 0.1 mmoles) in 0.1 M sodium acetate solution in small fractions. The solution was dialyzed against water (MWCO 10,000) until no free Gd³⁺ was detectable in the receiving vessel. The solution was lyophilized to yield 1.22 g of white powder (yield of polymer 81%). The number-average molecular weight of Gd³⁺-chelated polymeric conjugate was about 101,200 as measured by GPC. The compound contained 10.8% (w/w) of gadolinium.

Synthesis of PG-DTPA-Gd

PG-p-aminobenzyl-DTPA-Gd (PG-DTPA-Gd) was synthesized according to previously reported procedures. Wen X, Jackson E F, Price R E, et al. Synthesis and characterization of poly(L-glutamic acid) gadolinium chelate: a new biodegradable MRI contrast agent. Bioconjug Chem 2004; 15:1408-1415. Briefly, p-aminobenzyl-DTPA(t-butyl ester) (2.1 g, 2.79 mmol) was conjugated to PG (Mₙ, 41,400; 1 g, 7.75 mmol of carboxylic unit) in DMF using 1,3-diisopropylcarbodiimide (403 mg, 3.1 mmol) as the coupling agent. The t-butyl protecting groups were removed by treating with TFA at 4° C. overnight to give PG-DTPA. To chelate with Gd³⁺, a solution of GdCl₃.6H₂O in 0.1 M sodium acetate was added into a solution of PG-DTPA in 0.1 M sodium acetate (pH 5.5) in small fractions until free Gd³⁺ was detected. The solution was then dialyzed extensively against water (MWCO 10,000) and lyophilized to yield 1.22 g of white powder (81%). The compound contained 10.8% (w/w) of gadolinium.

Synthesis of PG-DTPA-Gd-IR783

PG-Benz-DTPA-Gd (90 mg, 0.698 mmol Glu) was dissolved in 2 mL of 0.1 M MES buffer. IR783-NH₂ (4.17 mg, 0.0045 mmol) dissolved in 200 uL of DMF was added to the PG-Bz-DTPA-Gd solution in the presence of EDC (10 mg, 0.005 mmol). This was stirred overnight at 4° C. while protected from light. The solution was filtered in 0.2 μm membrane filters and was dialyzed overnight with PBS buffer and water overnight at 4° C. Yield was 64.6 mg (72%).

Determination of Maximum Emission Wavelength

The fluorescence emission spectra of the synthesized contrast agent was obtained using a Spex Fluorolog spectrofluorimeter (Horiba Yvon Jobin, N.J.).

Determination of Relaxivity

Solutions of PG-Benz-DTPA-Gd-IR783 were prepared in water at gadolinium concentrations of 0.005, 0.01, 0.02, 0.04, 0.08, and 0.16 mM. Spin lattice (T₁) and spin-spin (T₂) relaxivities were measured at 4.7 Tesla on 4.7T Bruker Biospec 47/40USR (City, State) using inversion recovery and mutiecho T₂-weight pulse sequences. Relaxivities (R₁ or R₂ in mM⁻¹s⁻¹) were obtained from linear least square determination of the slopes of 1/T₁ vs [Gd] or 1/T₂ vs [Gd] plots.

Sentinel Lymph Node Identification

A group of 6 male athymic nude mice (NCI), 6-12 weeks old, were injected subcutaneously into the front paw with 10 μL of 0.002 mmol Gd/kg mouse or 5 nmol IR783/mouse of PG-benzDTPA-IR783 in PBS at pH 7.4. Optical images are taken before and at 5 minutes post-contrast and then, 10 μL of 1% (17.6 mM) isosulfan blue was injected into the same position as the PG-benzDTPA-IR783 was injected. After 5 minutes, an image-guided removal of lymph nodes and muscle was done. For histology, OCT-embedded tissue was cryo-sectioned at 10 μm thickness.

MR and Optical Imaging

Prior to imaging, mice were anesthetized with 1-2% isoflurane gas, and the entire animal was imaged for a maximum of 5 min at pre-contrast and at various times after subcutaneous injection of the contrast agent. For optical imaging, an IVIS imaging system (100 series) (Xenogen Corp., Alameda, Calif.) was used, while for MR imaging, a 4.7T Bruker Biospec 47/40USR MRI experimental scanner was used. During imaging, mice were maintained in an anesthetized state with 1.5% isoflurane.

Six mice were divided into two groups having 3 mice in each group. The first group was injected with 0.02 mmol Gd/kg mouse or 48 nmol IR783/mouse and the second group with 0.002 mmol Gd/kg mouse or 4.8 nmol IR783/mouse. Pre-contrast images of the mice were done at first in the optical imaging system and then the mice were imaged using MRI. T1-weighted image was set and after the baseline images were acquired, PG-benzDTPA-Gd-IR783 (0.02 mmol/kg or 0.002 mmol/kg) was rapidly injected into the front paw of the mice. Images were then taken every 3 minutes thereafter until 30 minutes. After the MR imaging, the mice were imaged using the optical imaging system and an image-guided removal of the sentinel lymph nodes and muscle was done. These tissues were frozen and cut into 10 um thick slices.

Total photon emissions from defined regions of interest within the optical images of each mouse were analyzed using the Living Image software (Xenogen Corporation, Alameda, Calif.), while imageJ software was used to analyze the MR images. The relative increase in signal intensity (SI) was calculated according to the formula ([Slpost−SIpre]/SIpre)×100%. For this analysis, the same region of interest (ROI) was drawn on the consecutive transaxial MR images. In the lymph nodes, the ROI was adapted to encompass as much of this structure as possible with maximum enhancement, and the same size of ROI was used in the pre-contrast images. All the results of data analysis were expressed as mean±SD. Significance of the differences of the data comparisons was assessed using a paired or unpaired Student t-test. A P value of less than 0.05 was taken to indicate statistical significance.

### Example 4

### Synthesis and Characterization of PG-benzDTPA-Gd-IR783

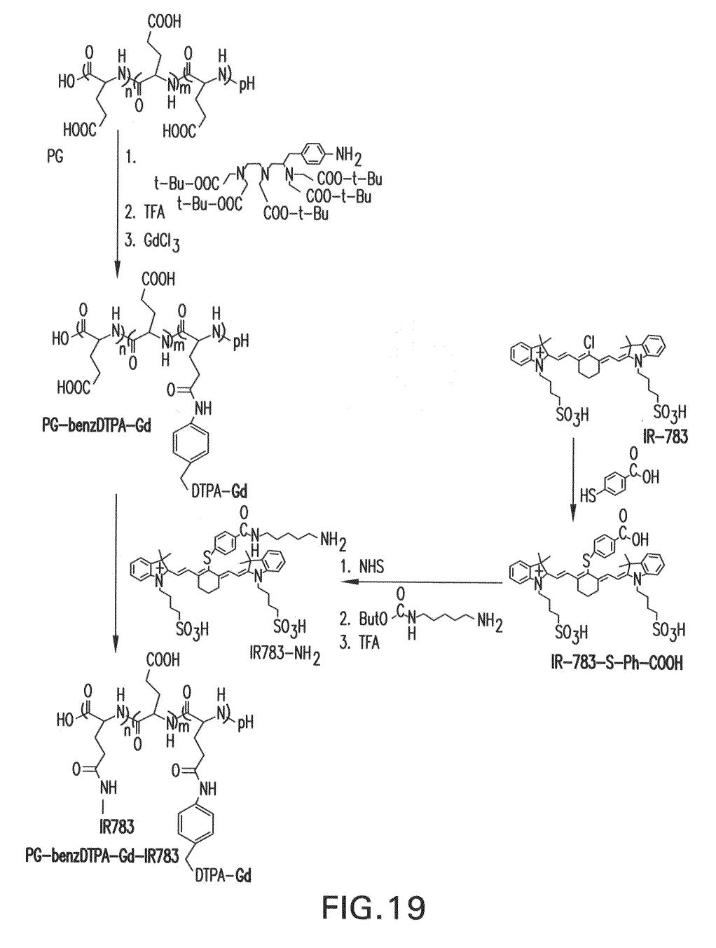

The synthetic scheme for the synthesis of PG-benzDTPA-Gd-IR783 is shown in FIG. 19. PG-benzDTPA-Gd was synthesized according to Wen X, et al. Bioconjugate Chem. 15: 1408-1415, 2004. IR783-NH₂ was conjugated to PG-benzDTPA-Gd using 1-ethyl-3-(3-dimethyl-aminopropyl) carbodiimide hydrochloride (EDC) as the coupling reagent. This conjugate was purified by dialysis against deionized water and by passing through PD-10 columns. The absence of small molecular weight contaminant was confirmed by gel permeation chromatography (GPC). Table 1 gives the summary of the physicochemical properties of the synthesized PG-benzDTPA-Gd and PG-benzDTPA-Gd-IR783. The starting PG has a molecular weight of 42,100. The molecular weight of the conjugated PG was calculated in terms of % Gd (w/w) and % IR783 (mol/mol). Percent Gd content by weight was determined using elemental analysis while % IR783 content was determined using fluorescence intensity. About 55 out of 274 glutamic acid units, or 0.2 mol/mol of COOH, were attached with Gd as measured by elemental analysis. About 3 IR783 units were attached to each PG chain.

TABLE 2

| | | | PG-Benz-DTPA- |
|---|---|---|---|
| | | PG-Benz-DTPA-Gd | Gd-IR783 |
| Mw calculated | | 60,080 | 62,813 |
| # COOH in PG | | 274 | 274 |
| # DTPA per PG | | 39 | 39 |
| % Gd (w/w) EA | | 10.83 | 10.40 |
| % Gd (w/w) calculated | | 9.25 | — |
| # DTPA per PG | | 39 | 39 |
| % Gd (w/w) EA | | 10.83 | 10.40 |
| % Gd (w/w) calculated | | 9.25 | — |
| % IR783 | | — | 1 |
| # IR783 per PG | | — | 3 |
| Relaxivity | (R1 mmol-1 s-1) | 8.89 | 13.23 |
| | (R2 mmol-1 s-1) | 24.07 | 39.08 |

Other physicochemical properties of the Gd³⁺-chelated PG polymers are also summarized in Table 2. The reported number average molecular weights were estimated from GPC analyses. For comparison, the theoretical number-average molecular weights calculated on the basis of starting molecular weight of PG and the degree of substitution are also listed. PG-benzDTPA-Gd-IR783 had greater relaxivity than that of small molecular weight DTPA-Gd, having T1 value of 4.8 mmol⁻¹s⁻¹ using 4.7T MRI experimental scanner (Table 2).

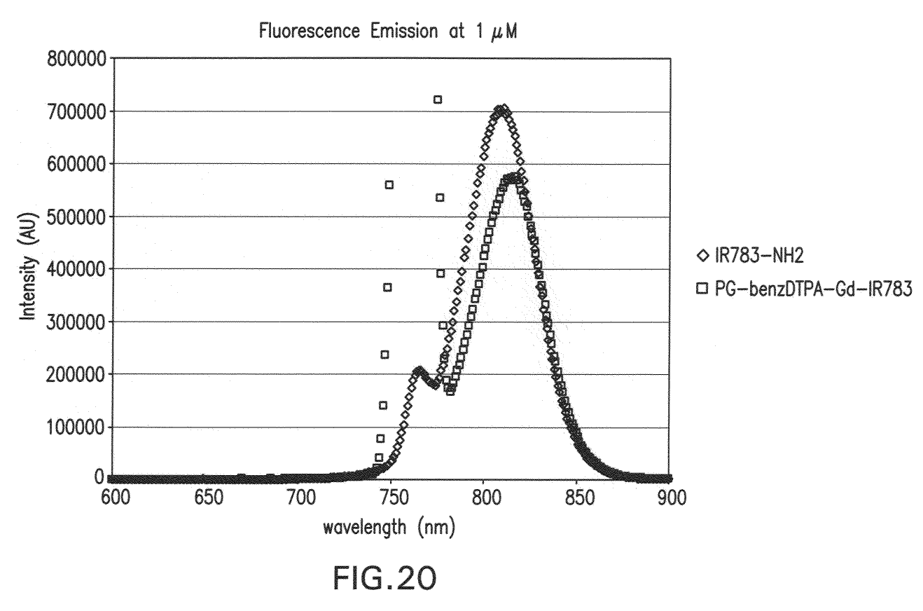

Comparison of fluorescence intensity of PG-benzDTPA-Gd-IR783 and IR783-NH₂ is presented in FIG. 20. A strong emission peak at around 805 nm was observed for IR783-NH₂, while PG-benzDTPA-Gd-IR783 has an emission at 814 nm.

Co-Localization of PG-benzDTPA-Gd-IR783 with Isosulfan Blue Dye

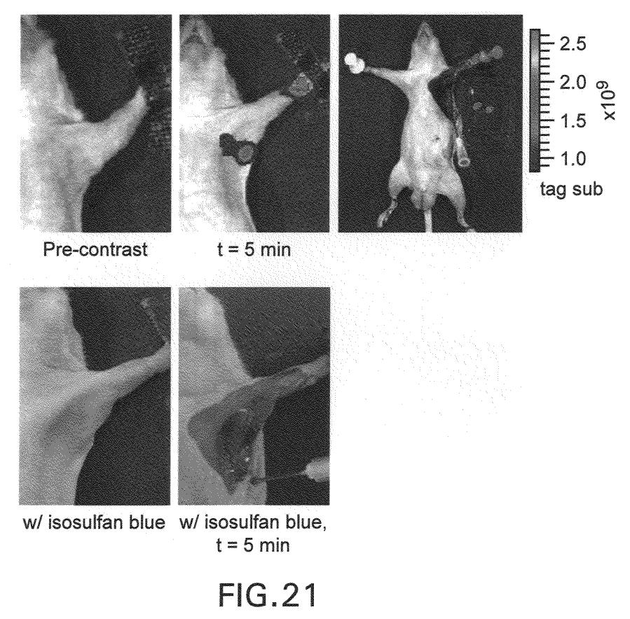

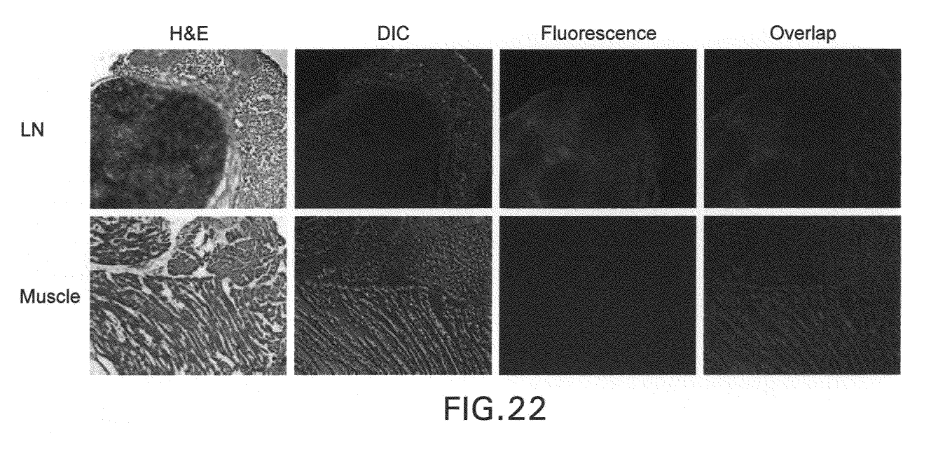

The PG-benzDTPA-Gd-IR783 has a maximal fluorescence emission at 814 nm, compared to IR783-NH₂ which is at 805 nm. When PG-benzDTPA-Gd-IR783 was injected subcutaneously into the front paw of the mouse, it entered the lymphatics and migrated within minutes to the axiliary and branchial lymph nodes. Co-injection at the same site with isosulfan blue, the gold standard of SLN mapping, resulted in co-localization of the NIR fluorescence signal and the blue dye (FIG. 21). Resection of these brightly fluorescent specimens was proved to be lymph nodes as conferred by hematoxylin and eosin (H&E) staining (FIG. 22). As a control, non-fluorescing muscle was also sectioned and imaged. As expected, muscle showed no fluorescence under the NIR fluorescent microscope.

MR and Optical Imaging Findings

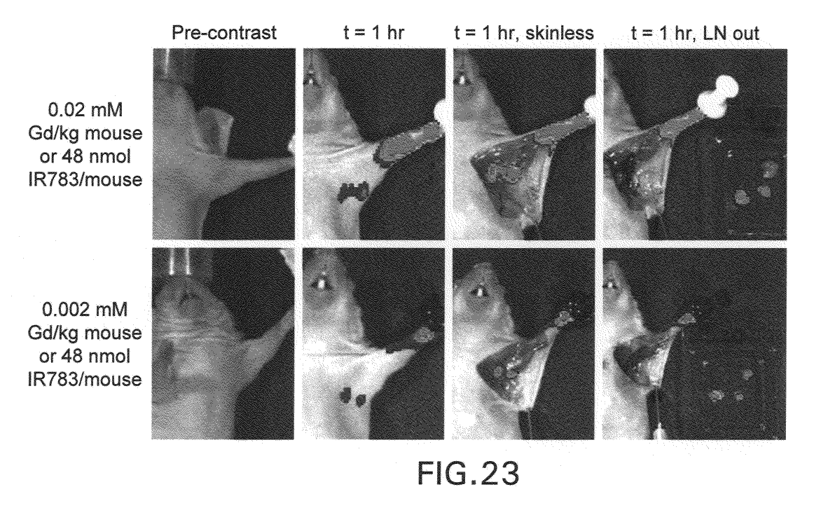

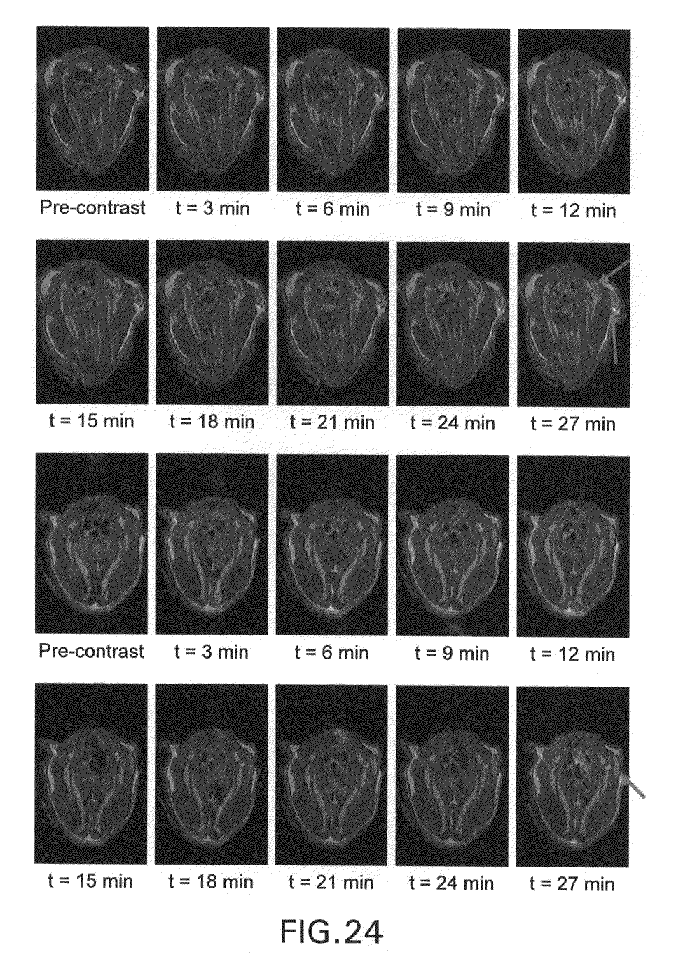

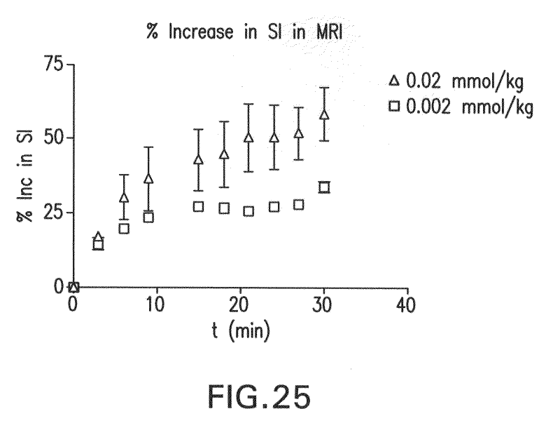

To demonstrate the ability of PG-benzDTPA-Gd-IR783 to act as a dual MR/optical imaging probe, we subcutaneously injected the agent into front paw of the mice (n=3) and obtained NIRF and MR images. FIG. 23 shows a representative example of NIRF images using 0.02 mmol Gd/kg or 48 nmol/mouse and 0.002 mmol Gd/kg or 4.8 nmol/mouse. The bright fluorescent images indicates uptake of the contrast agent into the axiliary and branchial lymph nodes. MR images also supports the NIRF images since branchial and axiliary lymph nodes indicated increase in signal enhancement post-contrast (FIGS. 24a and b). Calculation of the % increase in signal intensity reveals a concentration-dependent increase in signal enhancement, having a P-value <0.05 (FIG. 25). Examination of the 2 different concentrations showed that even at 0.002 mmol Gd/kg or 4.8 nmol/mouse, images can still be taken with great sensitivity.

Synthesis of PG-DTPA-Gd-NIR813

The synthetic scheme for the preparation of PG-DTPA-Gd-NIR813 is shown in FIG. 1B. PG-DTPA-Gd was dissolved. NIR813 (4.17 mg, 0.0045 mmol) dissolved in 200 μL of DMF was added to a solution of PG-DTPA-Gd (90 mg, 0.698 mmol Glu) in 0.1 M MES buffer (2 mL) in the presence of EDC (10 mg, 0.005 mmol). The reaction mixture was stirred at 4° C. overnight while protected from light, filtered through a 0.2-μm filter, dialyzed against PBS buffer and water sequentially, and lyophilized. Yield: 64.6 mg (72%). The conjugate contained about 4.4% NIR813 (w/w).

The physicochemical properties of PG-DTPA-Gd and PG-DTPA-Gd-NIR813 are summarized in Table 3. By GPC analysis, PG-DTPA-Gd-NIR813 had a number average molecular weight of 101,200. For comparison, the theoretical number-average molecular weight calculated on the basis of starting molecular weight of PG is also listed in Table 1. About 51 and 3 of the 274 glutamic acid units per PG chain were attached with DTPA-Gd and NIR813 dye, respectively. Table 3 shows the physico-chemical properties of PG-DTPA-Gd and PG-DTPA-Gd-IR783.

TABLE 3

| | | PG-DTPA-Gd | PG-DTPA-Gd-NIR813 |
|---|---|---|---|
| Molecular Weight^{a} | | 60,080 (274) | 62,813 (274) |
| % Gd (w/w)^{b} | | 10.83 | 10.40 |
| Number of DTPA per PG | | 39 | 39 |
| % NIR813 (w/w)^{c} | | — | 1 |
| Number of NIR813 per | | — | 3 |
| PG | | | |
| Relaxivity | (R1 mmol-1 s-1) | 8.89 | 13.23 |
| | (R2 mmol-1 s-1) | 24.07 | 39.08 |

^{a}Number average molecular weight calculated on the basis of starting molecular weight (42,100 Da) and the percentage of substitution.
^{b}Percentage of Gd by weighted was measured with elemental analysis.
^{c}Percentage of NIR813 measured spectrophotometrically.

The excitation/emission wavelengths were 766/798 nm for IR783 and 766/813 nm for NIR813 in methanol solution. Therefore NIR813 had a greater Stokes shift (47 nm) than IR783 (32 nm) did. A comparison of fluorescence emission spectra of NIR813 and PG-DTPA-Gd-NIR813 acquired at the same equivalent dye concentration is presented in FIG. 27. Both compounds had the same emission maximum of 813 nm when excited at 766 nm. However, the fluorescence intensity of PG-DTPA-Gd-NIR813 was reduced to approximately 44% of that of unconjugated NIR813, suggesting the presence of intramolecular interaction among NIR813 dyes attached to PG in aqueous solution. The presence of a shoulder peak at 765-775 nm supports that the dequenching effect observed with PG-DTPA-Gd-NIR813 was due to π-staggering of NIR813 in the polymer conjugate. Increasing the loading of NIR813 dye to more than 15 dye molecules per PG chain caused almost complete quenching of fluorescence signal (data not shown). Therefore, we used PG-DTPA-Gd-NIR813 containing on average 3 NIR813 molecules per polymer chain in our dual modality imaging studies. The fluorescence intensity of each PG-DTPA-Gd-NIR813 conjugate was approximately 32% stronger than one NIR813 molecule.

Relaxivity

Solutions of PG-DTPA-Gd-NIR813 were prepared in water at gadolinium concentrations of 0.005, 0.01, 0.02, 0.04, 0.08, and 0.16 mM. Spin lattice (T1) and spin-spin (T₂) relaxivities were measured at 4.7 Tesla on 4.7T Bruker Biospec (Bruker Biospin Corp., Billerica, Mass.) using inversion recovery and mutiecho T₂-weight pulse sequences. Relaxivities (R₁ or R₂ in mM⁻¹s⁻¹) were obtained from linear least square determination of the slopes of 1/T1 vs [Gd] or 1/T2 vs [Gd] plots.

Cell Line and Animals

Human DM14 squamous carcinoma cells were a soft agar clone derived from Tu167 cells (a gift from Dr. Clayman, MDACC). Cells were maintained at 37° C. in a humidified atmosphere containing 5% CO₂ in Dulbecco's modified Eagle's medium and nutrient mixture F-12 Ham (DMEM/F12) containing 10% fetal bovine serum (GIBCO, Grand Island, N.Y.).

All animal work was carried out in the Small Animal Imaging Facility at The University of Texas M. D. Anderson Cancer Center in accordance with institutional guidelines. For mice with lymph node metastases, 1×10⁶ DM14 cells suspended in 50 μL of HBSS were injected directly into the submucosa of the anterior tongue using a 1-ml tuberculin syringe (Hamilton Co.) and a 30-gauge needle in male athymic nude mice (n=3). By 20 days after inoculation, mice would die of malnutrition because the primary tumors prevented mice from food and water intake. Most mice would have developed metastases in the cervical lymph nodes by that time. Myers J N, Holsinger F C, Jasser S A, Bekele B N. Fidler I J. An orthotopic nude mouse model of oral tongue squamous cell carcinoma. Clin Cancer Res 2002; 8:293-298. Mice were used for imaging study on 10 days after tumor cell inoculation

MR and Optical Imaging

Prior to imaging, mice were anesthetized with 2% isoflurane gas in 1 l/min O₂ flow and during imaging, mice were maintained in an anesthetized state with 1.5% isoflurane. For optical imaging, an IVIS imaging system (100 series) (Xenogen Corp., Alameda, Calif.) was used with ICG filter (ex/em, 710-760/810-875 nm) sets. The field of view was 13.1 cm in diameter. The fluency rates for NIRF excitation light was 2 mW/cm². The camera settings included maximum gain, 2×2 binning, 640×480 pixel resolution and an exposure time of 0.8 sec. For MRI, a 4.7T Bruker Biospec scanner (Bruker Biospin Corp., Billerica, Mass.) was used. Axial and coronal images were obtained using a 950 mT/m, 5.7 cm inner diameter actively shielded gradient coil system (19,000 mT/m-s slew rate) and a 3.5 cm inner diameter volume radiofrequency coil. T1-weighted (TE=8.5 ms, TR=1000 ms) MR images were acquired with a 4×3 cm field of view, 1-mm section thickness, 0.25-mm gap, and a 256×192 matrix.

SLN Identification

A group of 6 male athymic nude mice (NCI, City, State), weighting 20-25 g each, were injected subcutaneously into the front paw with 10 μL of PG-DTPA-Gd-NIR813 (0.02 mmol Gd/kg, 48 nmol eq. NIR813/mouse) in PBS. Optical images were taken before and at 5 minutes post-contrast and then, 10 μL of 1% isosulfan blue (17.6 mM) was injected into the same sites as PG-DTPA-Gd-NIR813 was injected. Animals were killed 5 min later and the skin in the area where fluorescence signal was detected was removed to permit direct visual detection of the dye. Sentinel nodes noted for blue coloration under bright light were resected and imaged again with NIRF camera. Nodes were then processed for histologic evaluation.

Co-Localization of PG-DTPA-Gd-NIR813 with Isosulfan Blue Dye

When PG-DTPA-Gd-NIR813 was injected subcutaneously into the front paw of the mouse, it entered the lymphatics and migrated within minutes to the axiliary and branchial lymph nodes. Injection at the same site with isosulfan blue, the gold standard of SLN mapping, resulted in co-localization of the NIR fluorescence signal and the blue coloration (n=6, FIG. 3A-3D). These brightly fluorescent specimens were resected and proven to be lymph nodes histologically. No residual fluorescence signal was observed in the surrounding areas. Analysis of resected fluorescent tissues showed that PG-DTPA-Gd-NIR813 was completely trapped in SLN, but not in the surrounding tissues (FIG. 3E-H). Analysis also confirmed uptake of the contrast agent by lymph nodes.

Dual MR/Optical Imaging Detection of Axiliary and Branchial Lymph Nodes

Each mouse was injected subcutaneously in the left front paw with PG-DTPA-Gd-NIR813 at a dose of 0.02 mmol Gd/kg (48 nmol NIR813/mouse) or 0.002 mmol Gd/kg (4.8 nmol NIR813/mouse) (n=3/dose group). Pre-contrast images were obtained with both optical and MR imaging. T1-weighted MR images were then acquired every 3 minutes for 30 minutes post-contrast injection, after which the mice were imaged again with the NIRF camera. Sentinel lymph nodes were removed under NIRF guidance. The resected nodes were processed for histologic examinations.

For analysis of signal enhancement in sentinel nodes, the same region of interest (ROI), encompassing the whole enhanced axiliary lymph nodes, was drawn on the consecutive transaxial MR images. Image J software (http://rsb.info.nih.gov/ij/) was used to analyze the MR imaging data. The relative increase in MR signal intensity (SI %), calculated according to the formula SI %=(SIₚₒₛₜ−SIₚᵣₑ]/SIₚᵣₑ)×100%, was plotted as a function of time. SI % value at each time point was compared between two dose groups using an unpaired Student's t test with p<0.05 considered significant.

To examine whether sentinel auxiliary and branchial nodes could be detected with both MR and NIRF optical imaging, mice were given a single subcutaneous injection of PG-DTPA-Gd-NIR813 at a dose of 0.02 mmol Gd/kg as before or at a lower dose of 0.002 mmol Gd/kg. At both dose levels, the sentinel nodes were readily visualized with NIRF imaging. FIGS. 4A-D shows representative NIRF images acquired 1 hr after contrast injection at a lower dose of 0.002 mmol Gd/kg, which clearly revealed the uptake of the contrast agents in the auxiliary and branchial nodes. Resected lymph nodes showed bright fluorescence (FIG. 4D).

Both auxiliary and branchial nodes and their anatomical location were also identified as soon as 3 min after contrast injection on MR images at the high dose level (FIG. 4E). However, at the low dose level of 0.002 mmol Gd/kg, only the auxiliary node was visualized (FIG. 4F). Calculation of the % increase in MR signal intensity for the auxiliary nodes reveals a dose-dependent increase in signal enhancement. Signal intensities at a dose of 0.02 mmol Gd/kg were significantly higher than that at a dose of 0.02 mmol Gd/kg at each time points from 6 min post-injection over the 30 min study period (p<0.05, FIG. 5). MR signal intensity increased with time in a dose dependent manner.

Identification of Cervical Lymph Nodes and Detection of Metastases Following Imaging-Guided Nodal Resection

Mice were injected with PG-DTPA-Gd-NIR813 interstitially around the primary tumor at a dose of 0.02 mmol Gd/kg (48 mmol NIR813/mouse). Each mouse was imaged with optical and MRI before and at different times after contrast injection as described previously. At the end of the last imaging session (24 hr post contrast), sentinel nodes were removed under the guidance of NIRF imaging, and the resected tissues were processed for histologic examinations.

For histopathologic examinations, nodal tissues were embedded in optimal cutting temperature compound (OCT) (Sakura Finetek USA, Torrance, Calif.), snap-frozen, and cryosectioned into 10 μm slices, and stained with hematoxylin and eosin (H&E). Consecutive unstained sections were photographed on a Leica fluorescence microscope (Leica Microsystems, Bannockburn, Ill.). The microscope was equipped with a 75-W Xenon lamp, differential interference contrast (DIC) optical components, 775/845 nm (excitation/emission) filter sets (Chroma Technology, Brattleboro, Vt.), a Hamamatsu black and white chilled charge-coupled device camera (Hamamatsu Photonics K.K., Hamamatsu City, Japan), and Image-Pro Plus 4.5.1 software (Media Cybernetics, Silver Spring, Md.).

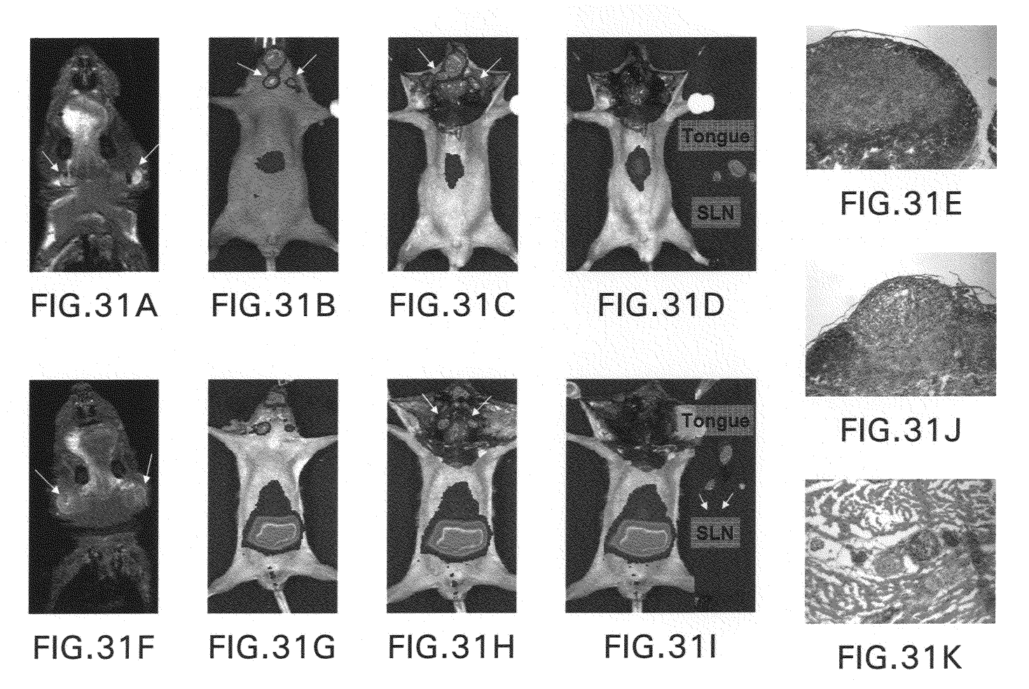

Whether dual MR/optical imaging using PG-DTPA-Gd-NIR813 could be used to characterize metastatic SLN preoperatively and postoperatively following imaging guided resection in an orthotopic head and neck tumor xenograft model was investigated. In mice without tumor, both MRI and NIRF imaging readily detected uptake of the contrast agent in the cervical lymph nodes after interstitial injection of PG-DTPA-Gd-NIR813 into the tongue of the mice at a dose of 0.02 mmol Gd/kg (FIG. 31A-31E). In mice with orthotopic human DM14 squamous carcinoma tumor grown in the tongue (n=3), all 6 sentinel nodes were visualized using NIRF imaging (FIG. 31G-I). However, 2 of the 6 nodes visualized with NIRF were not similarly identified by the MRI method. The pattern of enhancement of the remaining nodes revealed by MRI was different from that observed in normal cervical tissue: in general the lymph nodes showed less enhancement and the enhancement was located at the rim of the lymph nodes (compare FIGS. 31A vs. 31F). Histopathologic examination confirmed micrometastases in these nodes (FIG. 31J). Micrometastases were noted in the lumen of a vascular structure in the tongue of one of the tumor-bearing mice (FIG. 31K).

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

While the compositions and methods of this disclosure have been described in terms of specific embodiments, it will be apparent to those of skill in the art that variations may be applied to the compositions and/or methods and in the steps or in the sequence of steps of the methods described herein without departing from the concept, spirit and scope of the invention. All such similar substitutes and modifications apparent to those skilled in the art are deemed to be within the spirit, scope and concept of the invention.

1. A composition having the formula:

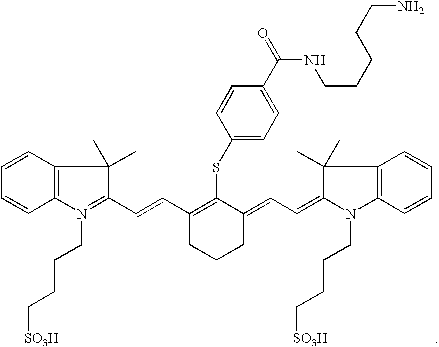

2. A composition comprising poly(L-glutamic acid) and a NIRF dye.

3. The composition of claim 2 wherein the NIRF dye is chosen from one or more of NIR813 and IR783.

4. The composition of claim 2 further comprising Gd-DTPA.

5. The composition of claim 2 wherein the NIRF dye is NIR813 and wherein the NIR813 is present at from about 1% to about 15% w/w.

6. The composition of claim 2 wherein the NIRF dye is NIR813 and further comprising Gd-DTPA.

7. The composition of claim 2 wherein the NIRF dye is NIR813 and is present at less than about 4% w/w and further comprising Gd-DTPA.

8. The composition of claim 2 wherein the NIRF dye is IR783 and further comprising benzDTPA-Gd.

9. The composition of claim 2 further comprising a therapeutic agent.

10. A method comprising:
providing to a plurality of cells an imaging agent comprising poly(L-glutamic acid), a NIRF dye; and
and imaging the cells to detect the imaging agent.

11. The method of claim 10, wherein the imaging agent further comprises a paramagnetic metal chelate.

12. The method of claim 10, wherein imaging the cells comprises measuring a NIRF signal.

13. The method of claim 10 wherein the plurality of cells are located in an animal subject.

14. The method of claim 11 wherein imaging comprises detecting with optical imaging or MR imaging or both.

15. A dual functional contrast agent comprising:
an MRI agent comprising Gadolinium conjugated with;
an optical imaging agent.

16. The agent of claim 15, wherein the optical imaging agent comprises a near-infrared fluorescence agent.

17. The agent of claim 16 wherein the near-infrared fluorescence agent comprises NIR813.

18. The agent of claim 15, further comprising a polymer having a molecular weight of at least 60 KDa.

19. The agent of claim 18 wherein the polymer comprises a poly(amino acid).

20. The agent of claim 19 wherein the poly (amino acid) comprises poly(L-glutamic acid).

21. The agent of claim 15, further comprising a chelating agent.

22. The agent of claim 21, wherein the chelating agent comprises DTPA.

23. The agent of claim 15, wherein the agent comprises PG-DTPA-Gd-NIR813.

24. A method of detecting cancer comprising:
injecting a dual functional contrast agent into a patient, wherein the dual functional contrast agent comprises:
an MRI agent conjugated with;
an optical imaging agent;
performing an MRI scan in the patient to detect the presence or absence of the contrast agent; and
performing an optical scan on the patient to detect the presence or absence of the contrast agent,
wherein presence of the contrast agent in a cell or tissue correlates with the presence of cancer in the cell or tissue.

25. A method according to claim 24, further comprising detecting the presence of the contrast agent in a lymph node cell or tissue.

26. A method according to claim 25, wherein the lymph node is a sentinel lymph node.

27. A method according to claim 24, comprising injecting and performing both scans prior to performing surgery on the patient.

28. A method according to claim 24, comprising injecting and performing both scans during surgery on the patient.

29. A method according to claim 24, wherein performing an optical scan comprises performing a near-infrared fluorescence scan.

30. A method according to claim 24, further comprising determining a cell or tissue to be treated or removed from the patient based on the MR scan and the optical scan.

31. A method of detecting cancer comprising:
injecting PG-DTPA-Gd-NIR813 into a patient;
detecting the presence or absence of Gd in a cell or tissue of the patient; and
detecting the presence or absence of NIR813 in a cell or tissue of the patient;
wherein the presence of Gd and NIR813 in a cell or tissue of the patient is indicative of cancer.

32. A method according to claim 31, wherein detecting the presence or absence of Gd comprises MRI.

33. A method according to claim 31, wherein detecting the presence or absence of nir813 comprises near-infrared fluorescence imaging.
